(19) United States
(12) Patent Application Publication
(10) Pub. No.: US 2026/0114796 A1
(43) Pub. Date: Apr. 30, 2026

(54) TRANSEPIDERMAL WATER LOSS AS AN ANAPHYLAXIS MONITORING TOOL

(71) Applicant: The Regents of the University of Michigan, Ann Arbor, MI (US)

(72) Inventors: Charles F. Schuler, IV, Ann Arbor, MI (US); Nicholas W. Lukacs, Ann Arbor, MI (US); James R. Baker, JR., Ann Arbor, MI (US)

(21) Appl. No.: 18/730,391

(22) PCT Filed: Jan. 19, 2023
(86) PCT No.: PCT/US2023/011135

§ 371 Date: Jul. 19, 2024

Related U.S. Application Data
(60) Provisional application No. 63/301,297, filed on Jan. 20, 2022.

(51) Int. Cl. A61B 5/00

(52) U.S. Cl. CPC A61B 5/411

(57) ABSTRACT

The present disclosure relates to systems and methods for detection of food allergy in a subject. In particular, the disclosure provides systems and methods for early detection of oral food challenge (OFC)-related anaphylaxis by measuring transepidermal water loss (TEWL).

### STATEMENT REGARDING RELATED APPLICATIONS

This application claims priority to U.S. Provisional Patent Application No. 63/301,297, filed Jan. 20, 2022, the entire contents of which are incorporated herein by reference for all purposes.

### FIELD

The present disclosure relates to systems and methods for detection of food allergy in a subject. In particular, the disclosure provides systems and methods for early detection of oral food challenge (OFC)-related anaphylaxis by measuring transepidermal water loss (TEWL).

### BACKGROUND

32 million Americans have food allergies, including nearly 11% of the population age 18 or older. Moreover, food allergy (FA) afflicts 5.6 million (8%) United States children and grows more common every year. FA leads to food anaphylaxis, a potentially fatal, whole-body allergic reaction that causes over 200,000 emergency room visits yearly in the United States. Skin and blood FA testing have false positive rates over 30%. For an accurate diagnosis, an oral food challenge (OFC) is needed; the person eats the food that may cause anaphylaxis and an allergist observes for a reaction. In very young children, the lack of expressive language delays anaphylaxis symptom detection, impacting challenge cessation and potentially increasing severity of anaphylaxis. The resulting fear of OFCs translates to unnecessary food avoidance, which severely impedes growth and nutrition and causes intense anxiety for parents and children. Accordingly, accurate methods for early detection of OFC-related anaphylaxis is sorely needed.

### SUMMARY

In some aspects, provided herein are methods. In some embodiments, provided herein are methods involving measuring transepidermal water loss (TEWL) from a subject. In some embodiments, TEWL is measured continuously. In some embodiments, TEWL is measured at one or more points in time following exposure to a suspected allergen. In some embodiments, measurements of TEWL can be used to determine treatment for a subject at risk of anaphylaxis. In some embodiments, measurements of TEWL can be used to predict anaphylaxis in a subject (e.g. in a subject not currently experiencing anaphylaxis). Accordingly, the methods provided herein permit early prediction of anaphylaxis, such as during an oral food challenge, thus improving upon the safety of oral food challenge tests.

In some aspects, provided herein are methods of predicting allergen-induced anaphylaxis in a subject. In some embodiments, provided herein is a method of predicting allergen-induced anaphylaxis in a subject, the method comprising exposing the subject to a suspected allergen, measuring a level of transepidermal water loss (TEWL) in the subject following exposure to the suspected allergen, and predicting anaphylaxis in the subject based upon the level of TEWL in the subject following exposure to the suspected allergen. In some embodiments, measuring a level of TEWL in the subject following exposure to the suspected allergen comprises continuously measuring TEWL in the subject for a duration of time. Any suitable duration of time may be used. In some embodiments, the duration of time is 15 seconds to 6 hours. In some embodiments, the duration of time is 15 seconds to 4 hours. In some embodiments, the duration of time is 15 seconds to 2 hours. In some embodiments, measuring a level of TEWL in the subject following exposure to the allergen comprises measuring a level of TEWL at at least one time point following exposure to the suspected allergen. In some embodiments, measuring a level of TEWL in the subject following exposure to the allergen comprises measuring a level of TEWL at a first point in time following exposure to the suspected allergen and measuring a level of TEWL at a second point in time following exposure to the suspected allergen, wherein the second point in time is after the first point in time.

In some embodiments, anaphylaxis is predicted in the subject when the level of TEWL at a time point following exposure to the suspected allergen is increased compared to a baseline level of TEWL in the subject; and/or the level of TEWL at a second time point following exposure to the suspected allergen is increased compared to a level of TEWL at first time point following exposure to the suspected allergen, wherein the second time point is after the first time point. For example, in some embodiments anaphylaxis is predicted in the subject when the level of TEWL at a time point following exposure to the suspected allergen is increased compared to a baseline level of TEWL in the subject. As another example, in some embodiments, anaphylaxis is predicted in the subject when the level of TEWL at a second time point following exposure to the suspected allergen is increased compared to a level of TEWL at first time point following exposure to the suspected allergen.

In some embodiments, the baseline level of TEWL is obtained from the subject no more than 4 hours before exposure to the suspected allergen. In some embodiments, the baseline level of TEWL is obtained from the subject no more than 1 hour before exposure to the suspected allergen.

In some embodiments anaphylaxis is predicted in the subject when the level of TEWL at a time point following exposure to the suspected allergen is increased compared to a baseline level of TEWL in the subject. In some embodiments, the time point (e.g. the time point following exposure to the suspected allergen) is no more than 4 hours after exposure to the suspected allergen. For example, in some embodiments the time point is no more than about 4 hours, no more than about 3.5 hours, no more than about 3 hours, no more than about 2.5 hours, no more than about 2 hours, no more than about 1.5 hours, no more than about 1 hour, or no more than about 30 minutes after exposure to the suspected allergen. In some embodiments, the time point no more than 2 hours after exposure to the suspected allergen. For example, in some embodiments the time point is no more than 2 hours, no more than 1.5 hours, no more than 1 hour, no more than 55 minutes, no more than 50 minutes, no more than 45 minutes, no more than 40 minutes, no more than 45 minutes, no more than 40 minutes, no more than 35 minutes, no more than 30 minutes, no more than 25 minutes, no more than 20 minutes, or less than 15 minutes after exposure to the suspected allergen. In some embodiments, the time point is about 1 minute, about 2 minutes, about 3 minutes, about 4 minutes, about 5 minutes, about 6 minutes, about 7 minutes, about 8 minutes, about 9 minutes, or about 10 minutes after exposure to the suspected allergen.

In some embodiments, the first time point is no more than 4 hours after exposure to the suspected allergen and the second time point is no more than 4 hours after the first time point. In some embodiments, the first time point is no more than 2 hours after exposure to the suspected allergen, and the second time point no more than 2 hours after the first time point. In some embodiments, the first time point is no more than 1 hour after exposure to the suspected allergen, and the second time point is no more than 1 hour after the first time point.

In some embodiments, anaphylaxis is predicted in the subject when the level of TEWL at the time point following exposure to the suspected allergen is increased by at least an absolute amount compared to the baseline level or when the level of TEWL at the second time point is increased by at least an absolute amount compared to the level of TEWL at the first time point. In some embodiments, the absolute amount is 1 g/m²/hr, 2 g/m²/hr, or 3 g/m²/hr.

In some embodiments, anaphylaxis is predicted when the level of TEWL at the time point following exposure to the suspected allergen is increased by at least 1 g/m²/hr compared to the baseline level and one or more symptoms of allergy are present in the subject, or the level of TEWL at the second time point is increased by at least 1 g/m²/hr compared to the level of TEWL at the first time point and one or more symptoms of allergy are present in the subject. For example, in some embodiments anaphylaxis is predicted when the level of TEWL at the time point following exposure to the suspected allergen is increased by at least 1 g/m²/hr compared to the baseline level and one or more symptoms of allergy are present in the subject. As another example, in some embodiments anaphylaxis is predicted when the level of TEWL at the second time point is increased by at least 1 g/m²/hr compared to the level of TEWL at the first time point and one or more symptoms of allergy are present in the subject. In some embodiments, the one or more symptoms of allergy are selected from hives, itching, sneezing, rhinorrhea, swelling, difficulty swallowing, shortness of breath, dizziness, nausea, and vomiting.

In some embodiments, anaphylaxis is predicted in the subject when the level of TEWL at the time point following exposure to the suspected allergen is at least doubled compared to the baseline level, or when the level of TEWL at the second time point is at least about double the level of TEWL at the first time point. In some embodiments, anaphylaxis is predicted in the subject when the level of TEWL at the time point following exposure to the suspected allergen is at least tripled compared to the baseline level, or when the level of TEWL at the second time point is at least about triple the level of TEWL at the first time point.

In some aspects, provided herein is a method of predicting allergen-induced anaphylaxis in a subject, the method comprising determining a baseline level of transepidermal water loss (TEWL) in the subject, exposing the subject to a suspected allergen, measuring a level of TEWL in the subject following exposure to the suspected allergen, and predicting anaphylaxis in the subject when the level of TEWL is increased following exposure to the suspected allergen compared to the baseline level. In some embodiments, the baseline level of TEWL is determined no more than 4 hours before exposing the subject to the suspected allergen. In some embodiments, the baseline level of TEWL in the subject is determined no more than 1 hour before exposing the subject to the suspected allergen. In some embodiments, the level of TEWL in the subject is measured no more than 4 hours after exposure to the suspected allergen. In some embodiments, the level of TEWL in the subject is measured no more than 2 hours after exposure to the suspected allergen.

In some embodiments, anaphylaxis is predicted in the subject when the level of TEWL following exposure to the suspected allergen is increased by at least an absolute amount compared to the baseline level. In some embodiments, the absolute amount is 1 g/m²/hr, 2 g/m²/hr, or 3 g/m²/hr. In some embodiments, anaphylaxis is predicted in the subject when the level of TEWL is at least doubled following exposure to the suspected allergen compared to the baseline level. In some embodiments, anaphylaxis is predicted in the subject when the level of TEWL is at least tripled following exposure to the suspected allergen compared to the baseline level. In some embodiments, anaphylaxis is predicted when the level of TEWL following exposure to the allergen is increased by at least 1 g/m²/hr compared to the baseline level and one or more symptoms of allergy are present in the subject. In some embodiments, the one or more symptoms of allergy are selected from hives, itching, sneezing, rhinorrhea, swelling, difficulty swallowing, shortness of breath, dizziness, nausea, and vomiting.

In some embodiments, measuring a level of TEWL in the subject following exposure to the suspected allergen comprises continuously measuring TEWL in the subject for a duration of time. In some embodiments, the duration of time is 15 seconds to 6 hours. In some embodiments, the duration of time is 15 seconds to 4 hours. In some embodiments, the duration of time is 15 seconds to 2 hours. In some embodiments, measuring a level of TEWL in the subject following exposure to the allergen comprises measuring a level of TEWL at at least one time point following exposure to the suspected allergen.

In some embodiments, provided herein is a method of predicting allergen-induced anaphylaxis in a subject, the method comprising exposing the subject to a suspected allergen, measuring a level of transepidermal water loss (TEWL) in the subject at a first time point following exposure to the suspected allergen, measuring a level of TEWL in the subject at a second time point following exposure to the suspected allergen, wherein the second time point is after the first time point, and predicting anaphylaxis in the subject when the level of TEWL at the second time point is increased compared to the level of TEWL at the first time point. In some embodiments, the method further comprises determining a baseline level of TEWL in the subject prior to exposing the subject to the suspected allergen.

In some embodiments, the first time point is no more than 4 hours after exposure to the suspected allergen, and the second time point is no more than 4 hours after the first time point. In some embodiments, the first time point is no more than 2 hours after exposure to the suspected allergen, and the second time point no more than 2 hours after the first time point. In some embodiments, the first time point is no more than 1 hour after exposure to the suspected allergen, and the second time point is no more than 1 hour after the first time point.

In some embodiments, anaphylaxis is predicted in the subject when the level of TEWL at the second time point is increased by at least an absolute amount compared to the level of TEWL at the first time point. In some embodiments, the absolute amount is 1 g/m²/hr, 2 g/m²/hr, or 3 g/m²/hr. In some embodiments, anaphylaxis is predicted in the subject when the level of TEWL at the second time point is at least about double the level of TEWL at the first time point. In some embodiments, anaphylaxis is predicted in the subject when the level of TEWL at the second time point is at least about triple the level of TEWL at the first time point. In some embodiments, anaphylaxis is predicted when the level of TEWL at the second time point is increased by at least 1 g/m²/hr compared to the level of TEWL at the first time point and one or more symptoms of allergy are present in the subject. In some embodiments, the one or more symptoms of allergy are selected from hives, itching, sneezing, rhinorrhea, swelling, difficulty swallowing, shortness of breath, dizziness, nausea, and vomiting.

In some embodiments, provided herein is a method of predicting allergen-induced anaphylaxis in a subject, the method comprising exposing the subject to a suspected allergen, continuously measuring levels of transepidermal water loss (TEWL) in the subject for a duration of time following exposure to the suspected allergen, and predicting anaphylaxis in the subject. In some embodiments, the method comprises predicting anaphylaxis in the subject when the level of TEWL at a time point following exposure to the suspected allergen is increased compared to a baseline level of TEWL in the subject; and/or the level of TEWL at a second time point following exposure to the suspected allergen is increased compared to a level of TEWL at first time point following exposure to the suspected allergen, wherein the second time point is after the first time point. For example, in some embodiments the method comprises predicting anaphylaxis in the subject when the level of TEWL at a time point following exposure to the suspected allergen is increased compared to a baseline level of TEWL in the subject. As another example, in some embodiments the method comprises predicting anaphylaxis when the level of TEWL at a second time point following exposure to the suspected allergen is increased compared to a level of TEWL at first time point following exposure to the suspected allergen. In some embodiments, the duration of time (e.g. the duration of time for which levels of TEWL are continuously measured) is 15 seconds to 6 hours. In some embodiments, the duration of time is 15 seconds to 4 hours. In some embodiments, the duration of time is 15 seconds to 2 hours.

In some embodiments, the method comprises obtaining a baseline level of TEWL in the subject. In some embodiments, the baseline level of TEWL is obtained from the subject no more than 4 hours before exposure to the suspected allergen. For example, in some embodiments the baseline level of TEWL is obtained from the subject no more than 1 hour before exposure to the suspected allergen.

In some embodiments, the method comprises predicting anaphylaxis in the subject when the level of TEWL at a time point following exposure to the suspected allergen is increased compared to a baseline level of TEWL in the subject. In some embodiments, the time point (e.g. the time point following exposure to the suspected allergen) is no more than 4 hours after exposure to the suspected allergen. In some embodiments, the time point no more than 2 hours after exposure to the suspected allergen.

In some embodiments, the method comprises predicting anaphylaxis when the level of TEWL at a second time point following exposure to the suspected allergen is increased compared to a level of TEWL at first time point following exposure to the suspected allergen. In some embodiments, the first time point is no more than 4 hours after exposure to the suspected allergen and the second time point is no more than 4 hours after the first time point. In some embodiments, the first time point is no more than 2 hours after exposure to the suspected allergen, and the second time point no more than 2 hours after the first time point. In some embodiments, the first time point is no more than 1 hour after exposure to the suspected allergen, and the second time point is no more than 1 hour after the first time point.

In some embodiments, anaphylaxis is predicted in the subject when the level of TEWL at a time point following exposure to the suspected allergen is increased by at least an absolute amount compared to the baseline level, or when the level of TEWL at the second time point is increased by at least an absolute amount compared to the level of TEWL at the first time point. In some embodiments, absolute amount is 1 g/m²/hr, 2 g/m²/hr, or 3 g/m²/hr.

In some embodiments, anaphylaxis is predicted in the subject when the level of TEWL at the time point following exposure to the suspected allergen is increased by at least 1 g/m²/hr compared to the baseline level and one or more symptoms of allergy are present in the subject, or the level of TEWL at the second time point is increased by at least 1 g/m²/hr compared to the level of TEWL at the first time point and one or more symptoms of allergy are present in the subject. For example, in some embodiments anaphylaxis is predicted in the subject when the level of TEWL at the time point following exposure to the suspected allergen is increased by at least 1 g/m²/hr compared to the baseline level and one or more symptoms of allergy are present in the subject. As another example, in some embodiments anaphylaxis is predicted in the subject when the level of TEWL at the second time point is increased by at least 1 g/m²/hr compared to the level of TEWL at the first time point and one or more symptoms of allergy are present in the subject. In some embodiments, the one or more symptoms of allergy are selected from hives, itching, sneezing, rhinorrhea, swelling, difficulty swallowing, shortness of breath, dizziness, nausea, and vomiting.

In some embodiments, anaphylaxis is predicted in the subject when the level of TEWL is at least doubled following exposure to the suspected allergen compared to the baseline level or when the level of TEWL at the second time point is at least about double the level of TEWL at the first time point. In some embodiments, anaphylaxis is predicted in the subject when the level of TEWL is at least tripled following exposure to the suspected allergen compared to the baseline level or when the level of TEWL at the second time point is at least about triple the level of TEWL at the first time point.

In some aspects, provided herein are methods involving measuring TEWL in a subject. In some embodiments, provided herein is a method comprising determining a baseline level of transepidermal water loss (TEWL) in a subject, exposing the subject to a suspected allergen, and measuring a level of TEWL in the subject after exposure to the suspected allergen. In some embodiments, the baseline level of TEWL in the subject is determined no more than 4 hours before exposing the subject to the suspected allergen. In some embodiments, the baseline level of TEWL in the subject is determined no more than 1 hour before exposing the subject to the suspected allergen. In some embodiments, the level of TEWL in the subject is measured no more than 4 hours after exposure to the suspected allergen. In some embodiments, the level of TEWL in the subject is measured no more than 2 hours after exposure to the suspected allergen.

In some embodiments, provided herein is a method comprising exposing a subject to a suspected allergen, measuring a level of transepidermal water loss (TEWL) in the subject at a first time point following exposure to the suspected allergen, and measuring a level of TEWL in the subject at a second time point following exposure to the suspected allergen. In some embodiments, the second time point is after the first time point. In some embodiments, the first time point is no more than 4 hours after exposure to the suspected allergen, and the second time point is no more than 4 hours after the first time point. In some embodiments, the first time point is no more than 2 hours after exposure to the suspected allergen, and the second time point no more than 2 hours after the first time point. In some embodiments, first time point is no more than 1 hour after exposure to the suspected allergen, and the second time point is no more than 1 hour after the first time point.

In some embodiments, provided herein is a method comprising exposing a subject to a suspected allergen, and continuously measuring transepidermal water loss in the subject for a duration of time following exposure to the suspected allergen. In some embodiments, the duration of time is 15 seconds to 6 hours. In some embodiments, the duration of time is 15 seconds to 4 hours. In some embodiments, the duration of time is 15 seconds to 2 hours. In some embodiments, the method further comprises determining a baseline level of TEWL in the subject prior to exposing the subject to the suspected allergen. In some embodiments, the baseline level of TEWL is determined no more than 4 hours before exposing the subject to the suspected allergen. In some embodiments, the baseline level of TEWL is determined no more than 1 hour before exposing the subject to the suspected allergen.

In some aspects, provided herein are methods of determining a treatment for a subject at risk of anaphylaxis. In some embodiments, provided herein is a method for determining a treatment for a subject at risk of anaphylaxis comprising exposing the subject to a suspected allergen, measuring a level of TEWL in the subject following exposure to the suspected allergen, and determining a treatment for the subject based upon the level of TEWL in the subject following exposure to the suspected allergen. In some embodiments, measuring a level of TEWL in the subject following exposure to the suspected allergen comprises continuously measuring TEWL in the subject for a duration of time. In some embodiments, the duration of time is 15 seconds to 6 hours. In some embodiments, the duration of time is 15 seconds to 4 hours. In some embodiments, the duration of time is 15 seconds to 2 hours. In some embodiments, measuring a level of TEWL in the subject following exposure to the allergen comprises measuring a level of TEWL at at least one time point following exposure to the suspected allergen.

In some embodiments, the treatment comprises an anti-anaphylactic agent when the level of TEWL at a time point following exposure to the suspected allergen is increased compared to a baseline level of TEWL in the subject; and/or the level of TEWL at a second time point following exposure to the suspected allergen is increased compared to a level of TEWL at first time point following exposure to the suspected allergen, wherein the second time point is after the first time point. For example, in some embodiments the treatment comprises an anti-anaphylactic agent when the level of TEWL at a time point following exposure to the suspected allergen is increased compared to a baseline level of TEWL in the subject. As another example, in some embodiments the treatment comprises an anti-anaphylactic agent when the level of TEWL at a second time point following exposure to the suspected allergen is increased compared to a level of TEWL at first time point following exposure to the suspected allergen.

In some embodiments, the baseline level of TEWL is obtained from the subject no more than 4 hours before exposure to the suspected allergen. In some embodiments, the baseline level of TEWL is obtained from the subject no more than 1 hour before exposure to the suspected allergen.

In some embodiments, the treatment comprises an anti-anaphylactic agent when the level of TEWL at a time point following exposure to the suspected allergen is increased compared to a baseline level of TEWL in the subject. In some embodiments, the time point (e.g. the time point following exposure to the suspected allergen is no more than 4 hours after exposure to the suspected allergen. In some embodiments, the time point is no more than 2 hours after exposure to the suspected allergen.

In some embodiments, the treatment comprises an anti-anaphylactic agent when the level of TEWL at a second time point following exposure to the suspected allergen is increased compared to a level of TEWL at first time point following exposure to the suspected allergen. In some embodiments, the first time point is no more than 4 hours after exposure to the suspected allergen and the second time point is no more than 4 hours after the first time point. In some embodiments, the first time point is no more than 2 hours after exposure to the suspected allergen, and the second time point no more than 2 hours after the first time point. In some embodiments, the first time point is no more than 1 hour after exposure to the suspected allergen, and the second time point is no more than 1 hour after the first time point.

In some embodiments, the treatment comprises an anti-anaphylactic agent when the level of TEWL at the time point following exposure to the suspected allergen is increased by at least an absolute amount compared to the baseline level or when the level of TEWL at the second time point is increased by at least an absolute amount compared to the level of TEWL at the first time point. In some embodiments, the absolute amount is 1 g/m²/hr, 2 g/m²/hr, or 3 g/m²/hr. In some embodiments, the treatment comprises an anti-anaphylactic agent when the level of TEWL at the time point following exposure to the suspected allergen is at least doubled compared to the baseline level or when the level of TEWL at the second time point is at least about double the level of TEWL at the first time point. In some embodiments, the treatment comprises an anti-anaphylactic agent when the level of TEWL at the time point following exposure to the suspected allergen is at least tripled compared to the baseline level or when the level of TEWL at the second time point is at least about triple the level of TEWL at the first time point.

Any suitable anti-anaphylactic agent may be selected. In some embodiments, the anti-anaphylactic agent comprises epinephrine.

For any of the methods described herein, the suspected allergen can be a suspected food allergen. In some embodiments, the subject is exposed to the suspected food allergen by oral consumption of the suspected food allergen. In some embodiments, the subject is a human. In some embodiments, the subject is a pediatric subject or an infant subject. In some embodiments, the subject is an adult subject. In some embodiments, the subject is at risk of having an anaphylactic response to the suspected allergen.

### DESCRIPTION OF THE DRAWINGS

FIG. 1A shows raw TEWL for one measurement. Data were collected in triplicate on the volar forearm of the subject. Note, optimal conditions require tewameter equilibration over 10 seconds after placement on the skin, shown by Measurement 1 (blue), which required about 10 seconds to reach stability. Measurements 2 and 3 have stable profiles and show calibrated measurements. FIG. 1B shows baseline TEWL from 3 healthy controls and 3 subjects with atopic dermatitis (AD) on uninvolved skin. Error bars show an individual's standard error of the mean. **p<0.01.

FIG. 2 shows TEWL measured on a hive's wheal and flare. Measured at baseline (normal skin) and within 10 minutes after histamine applied (flare, wheal).

FIGS. 3A-3B further exemplifies that TEWL rises during anaphylaxis. FIG. 3A shows mean TEWL values before the food challenge (pre-OFC), at the mid-point, and at the end for participants who did not react during a food challenge (i.e. non-reactors). Data were collected from 20 subjects. FIG. 3B shows mean TEWL levels pre-PFC, at the mid-point (anaphylaxis) or post-epinephrine treatment for subjects who experienced an allergic response during the OFC. For this group, the OFC was conducted using peanuts. Hives/vomiting occurred after dose 2-3, and TEWL rose before vomiting occurred. TEWL was measured on the volar forearm for all subjects. Error bars show standard error of the mean. TEWL was measured via tewameter with MPA_CTA Plus software. Means compared via ANOVA. *p<0.05, **p<0.01. NS=not significant.

FIGS. 4A-4C show results from a double blood oral food challenge conducted using 3 participants. Participants each experienced 3 placebo OFCs (i.e. non-allergens) and 6 reaction OFCs (milk, egg, peanut, cashew, and/or pistachio used as the allergen). Baseline TEWL, mid-OFC TEWL, and end-OFC levels were measured. FIG. 4A shows baseline TEWL, mid-OFC TEWL, and end-OFC TEWL levels for all subjects. FIG. 4B shows the mean net TEWL change for nonreactors vs. reactors at mid-OFC (e.g. at the time of anaphylaxis for reactors). There was a significant (p<0.05) difference between reactors/nonreactors. FIG. 4C shows the net change in TEWL between the start of the OFC and either the end of the 1-hr waiting period (non-reactors) or at least 30-60 minutes after epinephrine has stopped anaphylaxis for reactors (data shown for a subset of patients). Error bars show standard error of the mean. TEWL measured via tewameter on volar forearm with MPA_CTA Plus software. Means compared via ANOVA. *p<0.05, ns=not significant.

FIG. 5 shows the time course of TEWL changes for one patient who experienced anaphylaxis following cashew consumption. The figure illustrates an example of both non-reaction (e.g. placebo, shown in blue) and reaction (cashew reaction, red) TEWL values as a time-course for the same person.

FIG. 6A shows a comparison of the time to first symptom, time to TEWL increase by 1 unit (i.e. 1 g/m²/h), and time to epinephrine (i.e. time to clinical onset of anaphylaxis) in subjects that experienced a reaction during OFC. FIG. 6B shows the food dose at the first symptom, food dose at the TEWL rise by 1 unit, and the food dose at epinephrine delivery in subjects that experienced a reaction during OFC.

FIGS. 7A-7B show a comparison of net TEWL rise to skin prick test results for a subject following consumption of the same allergen. Net TEWL rise during a reaction food challenge does not correlate well with the existing skin prick test on the wheal (FIG. 7A) or flare (FIG. 7B). R² values are calculated using linear regression.

FIG. 8 shows a comparison of net TEWL rise to IgE measurements for a subject following consumption of the same allergen. Net TEWL rise during a reaction food challenge does not correlate well with IgE measurements. R² values are calculated using linear regression.

FIGS. 9A-9G show participants' intrinsic characteristics' influence on baseline TEWL. FIG. 9A shows baseline TEWL results from three body areas (volar forearm, supraclavicular neck, upper posterior torso over the scapula). FIG. 9B shows the difference between baseline TEWL and TEWL at food dose 2 or 3 during the OFC. FIGS. 9C, 9D, 9E, 9F, 9G, and 9H show baseline TEWL on volar forearm shown by age, sex, race, ethnicity, BMI, and AD status, respectively. FIG. 9C and FIG. 9G show simple linear regression results.

FIGS. 10A-10G show change in sTEWL during the OFCs. FIG. 10A and FIG. 10B show difference between baseline TEWL and TEWL at food dose 2 or 3 (nonreactors) or prior to epinephrine or other treatment (reactors) during the OFC. FIG. 10A shows all reactors and FIG. 10B shows only the reactors who required epinephrine. FIG. 10C shows the difference between baseline TEWL and TEWL at the end of the OFC. FIG. 10D shows the difference between baseline TEWL and TEWL at the end of the OFC for reactors only, separated by requiring epinephrine or not. FIG. 10E shows the difference between baseline TEWL and prior to epinephrine or other treatment versus CoFAR grade of anaphylaxis. FIG. 10F shows the difference between baseline TEWL and prior to epinephrine or other treatment versus AD status.

FIG. 10G shows the difference between baseline TEWL and TEWL at food dose 2 or 3 (nonreactors) or prior to epinephrine or other treatment (reactors) during the OFC shown by age, color-coded for reaction status. ****p<0.0001. ns=not significant.

FIGS. 11A-11K show baseline systemic immune markers. Baseline plasma results are shown for tryptase (FIG. 11A), IL-1β (FIG. 11B), IL-3 (FIG. 11C), IL-9 (FIG. 11D), IL-10 (FIG. 11E) IL-4 (FIG. 11F), IL-5 (FIG. 11G), IL-6, (FIG. 11H), IL-13 (FIG. 11I), TNF-α (FIG. 11J), and VEGF (FIG. 11K). All results are shown by reaction status. ns=not significant

FIGS. 12A-12K show change in systemic immune markers during OFC. Difference between post-OFC value and pre-OFC baseline result for the following markers: tryptase (FIG. 12A), IL-1β (FIG. 12B), IL-3 (FIG. 12C), IL-9 (FIG. 12D), IL-10 (FIG. 12E), IL-4 (FIG. 13F), IL-5 (FIG. 12G), IL-6 (FIG. 12H), IL-13 (FIG. 12I), TNF-α (FIG. 12J), and VEGF (FIG. 12K). All results are shown by reaction status. ***p<0.001. *p<0.05. ns=not significant.

FIGS. 13A-13C show timing of TEWL changes in relation to clinical events: FIG. 13A shows a representative time course of TEWL results for a reactive OFC (red) that led to anaphylaxis and a non-reactive challenge (blue). FIG. 13B shows time to first symptom, to a 1 g/m²/h rise in TEWL, and to epinephrine administration during OFCs resulting in anaphylaxis and requiring epinephrine administration. FIG. 13C shows food dose of first symptom, of a 1 g/m²/h rise in TEWL, and of epinephrine administration during OFCs resulting in anaphylaxis and requiring epinephrine administration. ***p<0.001. **p<0.01. *p<0.05.

FIGS. 14A-14C show results of continuous TEWL monitoring of the OFCs. FIG. 14A shows an example time course of a reactive OFC resulting in anaphylaxis (red) and a nonreactive OFC (blue). FIG. 14B shows the maximal net change in the mean TEWL value from the 2 minutes after any food dose during the OFC versus the 2 minutes prior to the same food dose. FIG. 14C shows sensitivity and specificity graphed visually for two TEWL stopping thresholds in combination with any single symptom. **p<0.01.

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “a peptide amphiphile” is a reference to one or more peptide amphiphiles and equivalents thereof known to those skilled in the art, and so forth.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “allergen” refers to any substance that is capable of inducing an allergic reaction in a subject. The term “allergen” as used herein is used in the broadest sense with relation to any allergen or component thereof, including food allergens (e.g. peanuts, tree nuts, eggs, milk, shellfish, wheat, etc.), environmental allergens (dust, pollen, pet dander, mold, insect bites, etc.), and the like. The term “allergen” is meant to include both whole allergens and allergen components. For example, an example of a whole allergen is a peanut. However, peanuts are known to contain 12 allergenic components, belonging to the Cupin superfamily (Ara h 1, 3), the Prolamin superfamily (Ara h 2, 6, 7, 9), the Profilin family (Ara h 5), and Bet v-1-related proteins (Ara h 8), as well as two additional families, Oleosin (Ara h 10,11) and Defensin (Ara h 12, 13). In some embodiments, the term “allergen” refers to a food allergen, such as peanuts or tree nuts or components thereof. The term “food allergen” refers to an allergen found in a food or beverage product. In some embodiments, an allergen is a medication (e.g. amoxicillin, penicillin, etc.). In some embodiments, the allergen is a drug. In some embodiments, the allergen is a venom.

As used herein, the term “anaphylaxis” refers to an allergic response characterized by one or more characteristics including narrowing of the airways, impaired breathing, a rapid, weak pulse, skin rash (e.g. hives), nausea, and/or vomiting. Anaphylaxis may be triggered within seconds or minutes of exposure to an allergen.

As used herein, the term “allergen-induced anaphylaxis” refers to anaphylaxis that occurs as a result of exposure to an allergen.

As used herein, the term “baseline level” refers to a level of transepidermal water loss in the subject prior to exposure to a suspected allergen. A baseline level may be obtained by averaging levels of TEWL across a duration of time or by averaging levels of TEWL obtained at multiple points in time prior to exposure to the suspected allergen.

As used herein, the terms “predict” or “predicting” when used in reference to anaphylaxis refers to determining the risk of anaphylaxis occurring in a subject. Predicting anaphylaxis in the subject does not necessarily mean that there is absolute certainty that the subject will experience anaphylaxis. Rather, predicting anaphylaxis indicates that the subject is at risk of experiencing anaphylaxis. For example, in some embodiments, “predicting anaphylaxis” refers to determining that there is at least a 50% likelihood (i.e. 50% chance) that the subject would experience anaphylaxis if exposure to the allergen were to continue and/or if no medical intervention occurred.

The term “transepidermal water loss” or “TEWL” are used interchangeably to refer to the quantity of condensed water that diffuses across a fixed area of stratum corneum to the skin surface per unit time. In some embodiments, TEWL is described in g/m²/h.

### DETAILED DESCRIPTION

In some aspects, provided herein are methods. In some embodiments, provided herein are methods involving measuring transepidermal water loss (TEWL) in a subject. In some embodiments, TEWL is measured continuously. In some embodiments, TEWL is measured at one or more points in time following exposure to a suspected allergen. For any of the methods described herein, TEWL can measured at any suitable area of the body, including but not limited to the arm (e.g. the volar forearm), the neck, or the back of the subject. In some embodiments, TEWL is measured at the arm (e.g. the volar forearm).

In some embodiments, level(s) of TEWL measured in the subject can be used to determine treatment for a subject at risk of anaphylaxis. For example, level(s) of TEWL in the subject can be used to determine treatment for a subject at risk of anaphylaxis during an oral food challenge. In some embodiments, TEWL in the subject can be used to predict anaphylaxis in a subject (e.g. in a subject not currently experiencing anaphylaxis). For example, in some embodiments TEWL in the subject can be used to predict anaphylaxis during an oral food challenge. Accordingly, the methods provided herein permit early prediction of anaphylaxis, such as during an oral food challenge, thus improving upon the safety of oral food challenge tests.

In some embodiments, the methods described herein, including methods comprising measuring TEWL in a subject, methods of predicting allergen-induced anaphylaxis in the subject, and methods of determining a treatment for a subject at risk of anaphylaxis, comprise exposing the subject to a suspected allergen. In some embodiments, the allergen is a food allergen. The food allergen may be a whole allergen (e.g. peanuts, milk, eggs) or an allergen component thereof. For example, for a peanut allergy the whole allergen would be a peanut, whereas an allergen component of a peanut may be selected from Ara h1, Ara h2, Ara h 3, Ara h 5, Ara h 6, Ara h 7, Ara h 8, Ara h 9, Ara h 10, Ara h 11, Ara h 12, or Ara h 13. As another example, for an egg allergy the whole antigen would be an egg, whereas an allergen component of an egg may be selected from ovomucoid (Gal d 1), ovalbumin (Gal d 2), ovotransferrin (Gal d 3), egg white lysozyme (Gal d 4), and ovomucin.

In some embodiments, the methods may be used to identify an allergy to a specific food allergen, and/or may be used to identify an allergy to a specific component within a whole allergen. Accordingly, in some embodiments exposing the subject to the suspected allergen involves ingestion of the suspected allergen by the subject (e.g. eating or drinking a composition comprising the suspected allergen). For example, the subject may ingest a whole suspected allergen (e.g. a peanut, milk, eggs, etc.). As another example, the subject may ingest an isolated allergic component of a whole allergen. In some embodiments, methods involve providing the allergen to the subject by performing an oral food challenge on the subject. In some embodiments, the oral food challenge comprises providing the subject with increasing doses of the suspected allergen at various time points. For example, the subject may consume/ingest a first, low dose of the suspected allergen at a first time point. After a set amount of time (typically about 15 minutes), the subject may ingest a second, higher dose of the suspected allergen. After another set amount of time (e.g. typically about another 15 minutes), the subject may ingest a third, higher dose of the suspected allergen, and so forth.

In some embodiments, the methods described herein comprise exposing the subject to the suspected allergen and measuring TEWL in the subject following exposure to the suspected allergen. In some embodiments, the methods comprise predicting anaphylaxis in the subject based upon TEWL in the subject. In some embodiments, the methods comprise determining a treatment for the subject based upon TEWL in the subject. In some embodiments, TEWL may be measured at one or more time points, and the level or amount of TEWL at those one or more time points may be used to predict anaphylaxis or determine a treatment for the subject. In some embodiments, a “time point” comprises a defined window of time during which TEWL is measured. The defined window of time may comprise any suitable duration of time, for example about 1 second, about 5 seconds, about 10 seconds, about 15 seconds, about 20 seconds, about 25 seconds, about 30 seconds, about 35 seconds, about 40 seconds, about 45 seconds, about 50 seconds, about 55 seconds, or about 60 seconds. For example, TEWL may be measured for about 30 seconds, and that 30 second window of time is referred to as a “time point”. In some embodiments, the level of TEWL at a given time point refers to an average level of TEWL across the window of time for that time point (e.g. the average level of TEWL across a 30 second span of time).

In some embodiments, TEWL is measured continuously. For embodiments wherein TEWL is measured continuously, an amount or level of TEWL at a time point or an average level of TEWL across a window of time may be selected. In some embodiments, an amount or level of TEWL at a time point or average level of TEWL across a window of time may be selected and used to predict anaphylaxis or determine a treatment for the subject. As with the above, the window of time may comprise any suitable duration of time (e.g. about 1 second, about 5 seconds, about 10 seconds, about 15 seconds, about 20 seconds, about 25 seconds, about 30 seconds, about 35 seconds, about 40 seconds, about 45 seconds, about 50 seconds, about 55 seconds, or about 60 seconds). For example, an average level of TEWL across a 30 second window of time (e.g. a “time point”) may be identified, and in some embodiments used to predict anaphylaxis or determine a treatment for the subject.

In some embodiments, the methods described herein comprise determining a baseline level of transepidermal water loss in the subject. For example, in some embodiments the methods comprise determining a baseline level of TEWL in the subject and measuring a level of TEWL in the subject following exposure to the suspected allergen. In some embodiments, the methods involve and predicting anaphylaxis in the subject or determining a treatment for the subject based upon a difference between post-exposure TEWL and baseline TEWL levels in the subject. For example, in some embodiments anaphylaxis is predicted in the subject (i.e. the subject is determined to be at risk of experiencing allergen-induced anaphylaxis) when the level of TEWL is increased following exposure to the suspected allergen compared to the baseline level.

In some aspects, provided herein are methods involving measuring TEWL in a subject. In some embodiments, the methods described herein involve measuring a baseline level of TEWL in the subject. In some embodiments, the methods involve measuring a baseline level of TEWL in the subject and measuring a level of TEWL in the subject after exposure to a suspected allergen. In some embodiments, the methods comprise measuring TEWL at multiple points in time after exposure to a suspected allergen. In some embodiments, TEWL is measured continuously for a suitable duration of time before and/or after exposure to the allergen. For example, in some embodiments TEWL is measured continuously to establish a baseline level of TEWL in the subject, and measured continuously during and after exposure to the suspected allergen to establish the subject's response to the suspected allergen. In some embodiments, TEWL is measured at multiple points in time before and/or after exposure to the suspected allergen. For example, in some embodiments TEWL is measured before exposure to the suspected allergen to generate a baseline level of TEWL in the subject, and TEWL is measured at multiple points in time following exposure to the suspected allergen to determine the subject's response to the suspected allergen. The level of TEWL at one or more time points after exposure to the suspected allergen can be used to predict anaphylaxis in the subject, such as by comparing the level of TEWL at a given tine point after exposure to the baseline level of TEWL, or by comparing the level of TEWL at a second time point after exposure to the suspected allergen to the level of TEWL at a first time point after exposure to the suspected allergen. The second time point is after the first time point.

In some embodiments, provided herein is a method comprising determining a baseline level of transepidermal water loss (TEWL) in a subject, exposing the subject to a suspected allergen, and measuring a level of TEWL in the subject after exposure to the suspected allergen. In some embodiments, the baseline level of TEWL in the subject is determined no more than 4 hours before exposing the subject to the suspected allergen. For example, in some embodiments the baseline level of TEWL in the subject is determined no more than about 4 hours, no more than about 3.5 hours, no more than about 3 hours, no more than about 2 hours, no more than about 1.5 hours, or no more than about 1 hour prior to exposure to the suspected allergen. In some embodiments, the baseline level of TEWL is obtained from the subject no more than 1 hour before exposure to the suspected allergen. For example, in some embodiments the baseline level of TEWL is obtained from the subject no more than about 1 hour, no more than about 50 minutes, no more than about 45 minutes, no more than about 40 minutes, no more than about 35 minutes, no more than about 30 minutes, no more than about 25 minutes, no more than about 20 minutes, no more than about 15 minutes, no more than about 10 minutes, no more than about 5 minutes, or about 1 minute prior to exposure to the suspected allergen.

In some embodiments, the level of TEWL in the subject is measured no more than 4 hours after exposure to the suspected allergen. For example, in some embodiments the level of TEWL in the subject is measured no more than about 4 hours, no more than about 3.5 hours, no more than about 3 hours, no more than about 2 hours, no more than about 1.5 hours, or no more than about 1 hour after exposure to the suspected allergen. In some embodiments, the level of TEWL in the subject is measured no more than 2 hours after exposure to the suspected allergen. For example, in some embodiments the level of TEWL in the subject is measured no more than 2 hours, no more than 1.5 hours, no more than 1 hour, no more than 55 minutes, no more than 50 minutes, no more than 45 minutes, no more than 40 minutes, no more than 45 minutes, no more than 40 minutes, no more than 35 minutes, no more than 30 minutes, no more than 25 minutes, no more than 20 minutes, or less than 15 minutes after exposure to the suspected allergen. In some embodiments, the level of TEWL in the subject is measured about 1 minute, about 2 minutes, about 3 minutes, about 4 minutes, about 5 minutes, about 6 minutes, about 7 minutes, about 8 minutes, about 9 minutes, or about 10 minutes after exposure to the suspected allergen.

In some embodiments, provided herein is a method comprising exposing a subject to a suspected allergen, measuring a level of transepidermal water loss (TEWL) in the subject at a first time point following exposure to the suspected allergen, and measuring a level of TEWL in the subject at a second time point following exposure to the suspected allergen. In some embodiments, the second time point is after the first time point. In some embodiments, the first time point is no more than 4 hours (e.g. no more than 4 hours, no more than 3.5 hours, no more than 3 hours, no more than 2.5 hours, no more than 2 hours, no more than 1.5 hours, no more than 1 hour) after exposure to the suspected allergen, and the second time point is no more than 4 hours (e.g. no more than about 4 hours, no more than about 3.5 hours, no more than about 3 hours, no more than about 2.5 hours, no more than about 2 hours, no more than about 1.5 hours, no more than about 1 hour) after the first time point. In some embodiments, the first time point is no more than 2 hours after exposure to the suspected allergen, and the second time point no more than 2 hours after the first time point. In some embodiments, first time point is no more than 1 hour after exposure to the suspected allergen, and wherein the second time point is no more than 1 hour after the first time point. For example, in some embodiments the first time point is no more than 2 hours, no more than 1.5 hours, no more than 1 hour, no more than 50 minutes, no more than 45 minutes, no more than 40 minutes no more than 35 minutes, no more than 30 minutes, no more than 25 minutes, no more than 20 minutes, no more than 15 minutes, no more than 10 minutes, no more than 5 minutes, about 4 minutes, about 3 minutes, about 2 minutes, or about 1 minute after exposure to the suspected allergen and the second time point is no more than 2 hours, no more than 1.5 hours, no more than 1 hour, no more than 50 minutes, no more than 45 minutes, no more than 40 minutes no more than 35 minutes, no more than 30 minutes, no more than 25 minutes, no more than 20 minutes, no more than 15 minutes, no more than 10 minutes, no more than 5 minutes, about 4 minutes, about 3 minutes, about 2 minutes, or about 1 minute after the first time point.

In some embodiments, provided herein is a method comprising exposing a subject to a suspected allergen, and continuously measuring transepidermal water loss in the subject for a duration of time following exposure to the suspected allergen. In some embodiments, the duration of time is 15 seconds to 6 hours. In some embodiments, the duration of time is 15 seconds to 4 hours. In some embodiments, the duration of time is 15 seconds to 2 hours. For example, in some embodiments the duration of time is about 15 seconds, about 30 seconds, about 60 seconds, about 2 minutes, about 3 minutes, about 4 minutes, about 5 minutes, about 6 minutes, about 7 minutes, about 8 minutes, about 9 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 70 minutes, about 80 minutes, about 90 minutes, about 100 minutes, about 110 minutes, or about 120 minutes. In some embodiments, the method further comprises determining a baseline level of TEWL in the subject prior to exposing the subject to the suspected allergen. In some embodiments, the baseline level of TEWL is determined no more than 4 hours (e.g. no more than about 4 hours, no more than about 3.5 hours, no more than about 3 hours, no more than about 2.5 hours, no more than about 2 hours, no more than about 1.5 hours, no more than about 1 hour) before exposing the subject to the suspected allergen. In some embodiments, the baseline level of TEWL is determined no more than 1 hour before exposing the subject to the suspected allergen.

In some aspects, provided herein are methods for predicting anaphylaxis in a subject. In some embodiments, methods for predicting anaphylaxis involve measuring transepidermal water loss in the subject, as described herein. TEWL may be measured in the subject before, during, and/or after exposure to a suspected allergen.

In some embodiments, provided herein is a method of predicting allergen-induced anaphylaxis in a subject, comprising exposing the subject to a suspected allergen, measuring a level of transepidermal water loss (TEWL) in the subject following exposure to the suspected allergen, and predicting anaphylaxis in the subject based upon the level of TEWL in the subject following exposure to the suspected allergen.

In some embodiments, measuring a level of TEWL in the subject following exposure to the suspected allergen comprises continuously measuring TEWL in the subject for a duration of time. For example, TEWL may be continuously measured in the subject, and if a level of TEWL at a given point in time during the continuous monitoring is increased compared to the baseline level, anaphylaxis is predicted in the subject (i.e. the subject is determined to be at risk of experiencing anaphylaxis). In some embodiments, TEWL is measured continuously in the subject during an entire oral food challenge. Any suitable duration of time may be used. In some embodiments, the duration of time is 15 seconds to 6 hours. In some embodiments, the duration of time is 15 seconds to 4 hours. In some embodiments, the duration of time is 15 seconds to 2 hours. For example, in some embodiments the duration of time is about 15 seconds, about 30 seconds, about 60 seconds, about 2 minutes, about 3 minutes, about 4 minutes, about 5 minutes, about 6 minutes, about 7 minutes, about 8 minutes, about 9 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 70 minutes, about 80 minutes, about 90 minutes, about 100 minutes, about 110 minutes, or about 120 minutes.

In some embodiments, measuring a level of TEWL in the subject following exposure to the allergen comprises measuring a level of TEWL at at least one time point following exposure to the suspected allergen. The at least one time point may be any suitable time point following exposure to the suspected allergen. In some embodiments, the at least one time point (e.g. the time point following exposure to the suspected allergen) is no more than 4 hours after exposure to the suspected allergen. For example, in some embodiments the at least one time point is no more than about 4 hours, no more than about 3.5 hours, no more than about 3 hours, no more than about 2.5 hours, no more than about 2 hours, no more than about 1.5 hours, no more than about 1 hour, or no more than about 30 minutes after exposure to the suspected allergen. In some embodiments, the time point no more than 2 hours after exposure to the suspected allergen. For example, in some embodiments the at least one time point is no more than 2 hours, no more than 1.5 hours, no more than 1 hour, no more than 55 minutes, no more than 50 minutes, no more than 45 minutes, no more than 40 minutes, no more than 45 minutes, no more than 40 minutes, no more than 35 minutes, no more than 30 minutes, no more than 25 minutes, no more than 20 minutes, or less than 15 minutes after exposure to the suspected allergen. In some embodiments, the at least one point in time is about 1 minute, about 2 minutes, about 3 minutes, about 4 minutes, about 5 minutes, about 6 minutes, about 7 minutes, about 8 minutes, about 9 minutes, or about 10 minutes after exposure to the suspected antigen. As described above, a “time point” may refer to a defined window of time (e.g. a 10 second window of time, a 20 second window of time, a 30 second window of time, etc.) and a level of TEWL at that time point may be an average level of TEWL across that window of time. For example, the subject may be exposed to the suspected allergen (e.g. by ingesting the suspected allergen) and at at least one point following exposure TEWL may be measured.

In some embodiments, the methods of predicting allergen-induced anaphylaxis in the subject comprise measuring a level of TEWL at multiple time points following exposure to the suspected allergen and predicting anaphylaxis in the subject (i.e. determining the risk of anaphylaxis in the subject) based upon the levels of TEWL at those multiple time points. For example, in some embodiments, provided herein is a method of predicting allergen-induced anaphylaxis in a subject, comprising exposing the subject to a suspected allergen, measuring a level of transepidermal water loss (TEWL) in the subject at a first time point following exposure to the suspected allergen, measuring a level of TEWL in the subject at a second time point following exposure to the suspected allergen, and predicting anaphylaxis in the subject when the level of TEWL at the second time point is increased compared to the level of TEWL at the first time point. In some embodiments, the second time point is after the first time point.

Any suitable spacing between the first time point and the second time point may be used. For example, the first and second time points may be separated by a time span of less than 1 minute, about 1 minute, about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, or more than 15 minutes. For example, TEWL may be measured at multiple time points after exposure, such as multiple time points selected from immediately after exposure (i.e. less than 30 seconds after exposure), about 1 minute, about 2 minutes, about 3 minutes, about 4 minutes, about 5 minutes, about 6 minutes, about 7 minutes, about 8 minutes, about 9 minutes, about 10 minutes, or more than 10 minutes following exposure. For example, a level of TEWL may be measured immediately after exposure, a level of TEWL may be measured about 5 minutes after exposure, and level of TEWL may be measured about 10 minutes after exposure. In some embodiments, anaphylaxis may be predicted in the subject if the level of TEWL at at least one time point is increased compared to the baseline level.

In some embodiments, measuring a level of transepidermal water loss (TEWL) in the subject following exposure to the suspected allergen comprises measuring a level of TEWL at a first time point following exposure to the suspected allergen, and measuring a level of TEWL in the subject at a second time point following exposure to the suspected allergen. In some embodiments, the second time point is after the first time point. In some embodiments, the first time point is no more than 4 hours (e.g. no more than 4 hours, no more than 3.5 hours, no more than 3 hours, no more than 2.5 hours, no more than 2 hours, no more than 1.5 hours, no more than 1 hour) after exposure to the suspected allergen, and the second time point is no more than 4 hours (e.g. no more than about 4 hours, no more than about 3.5 hours, no more than about 3 hours, no more than about 2.5 hours, no more than about 2 hours, no more than about 1.5 hours, no more than about 1 hour) after the first time point. In some embodiments, the first time point is no more than 2 hours after exposure to the suspected allergen, and the second time point no more than 2 hours after the first time point. In some embodiments, first time point is no more than 1 hour after exposure to the suspected allergen, and wherein the second time point is no more than 1 hour after the first time point. For example, in some embodiments the first time point is no more than 2 hours, no more than 1.5 hours, no more than 1 hour, no more than 50 minutes, no more than 45 minutes, no more than 40 minutes no more than 35 minutes, no more than 30 minutes, no more than 25 minutes, no more than 20 minutes, no more than 15 minutes, no more than 10 minutes, no more than 5 minutes, or about 1 minute after exposure to the suspected allergen and the second time point is no more than 2 hours, no more than 1.5 hours, no more than 1 hour, no more than 50 minutes, no more than 45 minutes, no more than 40 minutes no more than 35 minutes, no more than 30 minutes, no more than 25 minutes, no more than 20 minutes, no more than 15 minutes, no more than 10 minutes, no more than 5 minutes, or about 1 minute after the first time point.

In some embodiments, anaphylaxis is predicted in the subject when the level of TEWL at a time point following exposure to the suspected allergen is increased compared to a baseline level of TEWL in the subject; and/or the level of TEWL at the second time point following exposure to the suspected allergen is increased compared to a level of TEWL at the first time point following exposure to the suspected allergen. For example, in some embodiments anaphylaxis is predicted in the subject when the level of TEWL at a time point following exposure to the suspected allergen is increased compared to a baseline level of TEWL in the subject. As another example, in some embodiments, anaphylaxis is predicted in the subject when the level of TEWL at a second time point following exposure to the suspected allergen is increased compared to a level of TEWL at first time point following exposure to the suspected allergen.

In some embodiments, anaphylaxis is predicted in the subject when the level of TEWL following exposure to the suspected allergen is increased by at least an absolute amount compared to the baseline level. In some embodiments, anaphylaxis is predicted in the subject when the level of TEWL at a second time point following exposure to the suspected allergen is increased by at least an absolute amount compared to a level of TEWL at first time point following exposure to the suspected allergen. In some embodiments, the absolute amount is at least 0.5 g/m²/h, at least 0.6 g/m²/h, 0.7 g/m²/h, 0.8 g/m²/h, 0.9 g/m²/h, 1.0 g/m²/h, 1.1 g/m²/h, 1.2 g/m²/h, 1.3 g/m²/h, 1.4 g/m²/h, 1.5 g/m²/h, 1.6 g/m²/h, 1.7 g/m²/h, 1.8 g/m²/h, 1.9 g/m²/h, 2.0 g/m²/h, 2.1 g/m²/h, 2.2 g/m²/h, 2.3 g/m²/h, 2.4 g/m²/h, 2.5 g/m²/h, 2.6 g/m²/h, 2.7 g/m²/h, 2.8 g/m²/h, 2.9 g/m²/h, or at least 3.0 g/m²/h. For example, anaphylaxis may be predicted in the subject when the level of TEWL is increased by at least 0.5 g/m²/h, at least 0.6 g/m²/h, 0.7 g/m²/h, 0.8 g/m²/h, 0.9 g/m²/h, 1.0 g/m²/h, 1.1 g/m²/h, 1.2 g/m²/h, 1.3 g/m²/h, 1.4 g/m²/h, 1.5 g/m²/h, 1.6 g/m²/h, 1.7 g/m²/h, 1.8 g/m²/h, 1.9 g/m²/h, 2.0 g/m²/h, 2.1 g/m²/h, 2.2 g/m²/h, 2.3 g/m²/h, 2.4 g/m²/h, 2.5 g/m²/h, 2.6 g/m²/h, 2.7 g/m²/h, 2.8 g/m²/h, 2.9 g/m²/h, or at least 3.0 g/m²/h following exposure to the suspected allergen compared to the baseline level. In some embodiments, the absolute amount is 1.0 g/m²/h, 2.0 g/m²/h, or 3.0 g/m²/h. As another example, in some embodiments anaphylaxis is predicted in the subject when the level of TEWL is increased by at least 0.5 g/m²/h, at least 0.6 g/m²/h, 0.7 g/m²/h, 0.8 g/m²/h, 0.9 g/m²/h, 1.0 g/m²/h, 1.1 g/m²/h, 1.2 g/m²/h, 1.3 g/m²/h, 1.4 g/m²/h, 1.5 g/m²/h, 1.6 g/m²/h, 1.7 g/m²/h, 1.8 g/m²/h, 1.9 g/m²/h, 2.0 g/m²/h, 2.1 g/m²/h, 2.2 g/m²/h, 2.3 g/m²/h, 2.4 g/m²/h, 2.5 g/m²/h, 2.6 g/m²/h, 2.7 g/m²/h, 2.8 g/m²/h, 2.9 g/m²/h, or at least 3.0 g/m²/h at the second time point compared to the level of TEWL at the first time point. In some embodiments, the absolute amount is 1.0 g/m²/h, 2.0 g/m²/h, or 3.0 g/m²/h.

In some embodiments, anaphylaxis is predicted in the subject when the level of TEWL is at least doubled following exposure to the suspected allergen compared to the baseline level. In some embodiments, anaphylaxis is predicted in the subject when the level of TEWL at the second time point is at least double the level of TEWL at the first time point. For example, if baseline level of TEWL in the subject was about 7 g/m²/h, anaphylaxis may be predicted in the subject if the level of TEWL following exposure to the allergen was at least about 14 g/m²/h. In some embodiments, anaphylaxis is predicted in the subject when the level of TEWL is at least tripled following exposure to the suspected allergen compared to the baseline level. In some embodiments, anaphylaxis is predicted in the subject when the level of TEWL is at least tripled at the second time point compared to the level of TEWL at the first time point.

In some embodiments, the method further comprises determining whether one or more symptoms of allergy are present in the subject. For example, in some embodiments the method comprises determining whether one or more symptoms of allergy selected from hives, itching, sneezing, rhinorrhea, swelling, difficulty swallowing, shortness of breath, dizziness, nausea, and vomiting are present in the subject. In some embodiments, anaphylaxis is predicted when the level of TEWL following exposure to the allergen is increased by at least 1 g/m²/hr compared to the baseline level and one or more symptoms of allergy are present in the subject. In some embodiments, anaphylaxis is predicted when the level of TEWL at the second time point is increased by at least 1 g/m²/hr compared to the level of TEWL at the first time point and one or more symptoms of allergy are present in the subject.

In some embodiments, levels of TEWL in the subject is measured continuously. For example, in some embodiments provided herein is a method of predicting allergen-induced anaphylaxis in the subject that involves continuously measuring TEWL in the subject. In some embodiments, TEWL is continuously measured, at levels of TEWL at one or more time points are selected to aid in the prediction of anaphylaxis in the subject. In some embodiments TEWL is measured continuously before, during, and/or after exposure to the suspected allergen.

In some embodiments, provided herein is a method of predicting allergen-induced anaphylaxis in a subject, the method comprising exposing the subject to a suspected allergen, continuously measuring levels of transepidermal water loss (TEWL) in the subject following exposure to the suspected allergen, and predicting anaphylaxis in the subject based upon the level of TEWL at one or more time points selected from the continuous monitoring. For example, a level at a given time point during the continuous measurement may be identified, and that level may be used to predict anaphylaxis in the subject. For example, if the level at that given time point is increased (e.g. increased by an absolute amount) compared to baseline, anaphylaxis may be predicted. As another example, anaphylaxis may be predicted if the level at that given time point is increased (e.g. increased by an absolute amount) compared to a level at an earlier time point following exposure to the suspected allergen. For example, TEWL may be measured continuously in the subject, and if a level of TEWL at 5 minutes after exposure to the suspected allergen is increased compared to a level of TEWL at 1 minute after exposure to the suspected allergen, anaphylaxis may be predicted.

In some embodiments, anaphylaxis is predicted in the subject when the level of TEWL at a given point in time following exposure to the suspected allergen is increased compared to a baseline level of TEWL in the subject. The baseline level is obtained prior to exposure to the suspected allergen. In some embodiments, anaphylaxis is predicted in the subject when the level of TEWL at a given point in time following exposure is increased by at least an absolute amount compared to the baseline level, the level of TEWL is at least doubled following exposure to the suspected allergen compared to the baseline level, or the level of TEWL is at least tripled following exposure to the suspected allergen compared to the baseline level. For example, in some embodiments anaphylaxis is predicted in the subject when the level of TEWL at a given time point following exposure is increased by at least an absolute amount compared to the baseline level. In some embodiments, the absolute amount is least 0.5 g/m²/h, at least 0.6 g/m²/h, 0.7 g/m²/h, 0.8 g/m²/h, 0.9 g/m²/h, 1.0 g/m²/h, 1.1 g/m²/h, 1.2 g/m²/h, 1.3 g/m²/h, 1.4 g/m²/h, 1.5 g/m²/h, 1.6 g/m²/h, 1.7 g/m²/h, 1.8 g/m²/h, 1.9 g/m²/h, 2.0 g/m²/h, 2.1 g/m²/h, 2.2 g/m²/h, 2.3 g/m²/h, 2.4 g/m²/h, 2.5 g/m²/h, 2.6 g/m²/h, 2.7 g/m²/h, 2.8 g/m²/h, 2.9 g/m²/h, or at least 3.0 g/m²/h. In some embodiments, the absolute amount is 1.0 g/m²/h, 2.0 g/m²/h, or 3.0 g/m²/h. In some embodiments, anaphylaxis is predicted when the level of TEWL is at least doubled following exposure to the suspected allergen compared to the baseline level. In some embodiments, anaphylaxis is predicted when the level of TEWL is at least tripled following exposure to the suspected allergen compared to the baseline level.

In some embodiments, anaphylaxis is predicted in the subject when the level of TEWL at a second time point is increased compared to the level at first time point. The second time point is after the first time point. For example, anaphylaxis may be predicted in the subject when the level of TEWL at a given time point is increased by at least an absolute amount compared to the level of TEWL at the earlier time point, the level of TEWL at the given time point is at least about double the level of TEWL at the earlier time point, or the level of TEWL at the given time point is at least about triple the level of TEWL at the earlier time point. For example, in some embodiments anaphylaxis is predicted in the subject when the level of TEWL at a given time point following exposure is increased by at least an absolute amount compared to the level of TEWL at an earlier time point. In some embodiments, the absolute amount is least 0.5 g/m²/h, at least 0.6 g/m²/h, 0.7 g/m²/h, 0.8 g/m²/h, 0.9 g/m²/h, 1.0 g/m²/h, 1.1 g/m²/h, 1.2 g/m²/h, 1.3 g/m²/h, 1.4 g/m²/h, 1.5 g/m²/h, 1.6 g/m²/h, 1.7 g/m²/h, 1.8 g/m²/h, 1.9 g/m²/h, 2.0 g/m²/h, 2.1 g/m²/h, 2.2 g/m²/h, 2.3 g/m²/h, 2.4 g/m²/h, 2.5 g/m²/h, 2.6 g/m²/h, 2.7 g/m²/h, 2.8 g/m²/h, 2.9 g/m²/h, or at least 3.0 g/m²/h. In some embodiments, the absolute amount is 1.0 g/m²/h, 2.0 g/m²/h, or 3.0 g/m²/h.

In some embodiments, anaphylaxis is predicted in the subject when the level of TEWL at the second time point following exposure is increased by at least an absolute amount compared to the first time point, the level of TEWL at the second time point is at least doubled compared to the level of TEWL at the first time point, or the level of TEWL at the second time point is at least tripled compared to the level of TEWL at the first time point.

In some embodiments, the method further comprises determining whether one or more symptoms of allergy are present in the subject. For example, in some embodiments the method comprises determining whether one or more symptoms of allergy selected from hives, itching, sneezing, rhinorrhea, swelling, difficulty swallowing, shortness of breath, dizziness, nausea, and vomiting are present in the subject. In some embodiments, anaphylaxis is predicted when the level of TEWL following exposure to the allergen is increased by at least 1 g/m²/hr compared to the baseline level, and one or more symptoms of allergy are present in the subject. In some embodiments, anaphylaxis is predicted when the level of TEWL at the second time point is increased by at least 1 g/m²/hr compared to the first time point, and one or more symptoms of allergy are present in the subject.

In some embodiments, provided herein are methods of determining a treatment for a subject at risk of anaphylaxis. In some embodiments, methods for determining a treatment for a subject at risk of anaphylaxis involve measuring transepidermal water loss in the subject, as described herein. TEWL may be measured in the subject before, during, and/or after exposure to a suspected allergen. In some embodiments, provided herein is a method of determining a treatment for a subject at risk of anaphylaxis, comprising exposing the subject to a suspected allergen, measuring a level of TEWL in the subject following exposure to the suspected allergen, and determining a treatment for the subject based upon the level of TEWL in the subject following exposure to the suspected allergen.

As described above, measuring a level of TEWL in the subject following exposure to the suspected allergen comprises continuously measuring TEWL in the subject for a duration of time. For example, TEWL may be continuously measured in the subject, and if a level of TEWL at a given point in time during the continuous monitoring is increased compared to the baseline level, anaphylaxis is predicted in the subject (i.e. the subject is determined to be at risk of experiencing anaphylaxis). In some embodiments, TEWL is measured continuously in the subject during an entire oral food challenge. Any suitable duration of time may be used. In some embodiments, the duration of time is 15 seconds to 6 hours. In some embodiments, the duration of time is 15 seconds to 4 hours. In some embodiments, the duration of time is 15 seconds to 2 hours. For example, in some embodiments the duration of time is about 15 seconds, about 30 seconds, about 60 seconds, about 2 minutes, about 3 minutes, about 4 minutes, about 5 minutes, about 6 minutes, about 7 minutes, about 8 minutes, about 9 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 70 minutes, about 80 minutes, about 90 minutes, about 100 minutes, about 110 minutes, or about 120 minutes.

In some embodiments, measuring a level of TEWL in the subject following exposure to the allergen comprises measuring a level of TEWL at at least one time point following exposure to the suspected allergen. The at least one time point may be any suitable time point following exposure to the suspected allergen. In some embodiments, the at least one time point (e.g. the time point following exposure to the suspected allergen) is no more than 4 hours after exposure to the suspected allergen. For example, in some embodiments the at least one time point is no more than about 4 hours, no more than about 3.5 hours, no more than about 3 hours, no more than about 2.5 hours, no more than about 2 hours, no more than about 1.5 hours, no more than about 1 hour, or no more than about 30 minutes after exposure to the suspected allergen. In some embodiments, the time point no more than 2 hours after exposure to the suspected allergen. For example, in some embodiments the at least one time point is no more than 2 hours, no more than 1.5 hours, no more than 1 hour, no more than 55 minutes, no more than 50 minutes, no more than 45 minutes, no more than 40 minutes, no more than 45 minutes, no more than 40 minutes, no more than 35 minutes, no more than 30 minutes, no more than 25 minutes, no more than 20 minutes, or less than 15 minutes after exposure to the suspected allergen. In some embodiments, the at least one point in time is about 1 minute, about 2 minutes, about 3 minutes, about 4 minutes, about 5 minutes, about 6 minutes, about 7 minutes, about 8 minutes, about 9 minutes, or about 10 minutes after exposure to the suspected antigen. As described above, a “time point” may refer to a defined window of time (e.g. a 10 second window of time, a 20 second window of time, a 30 second window of time, etc.) and a level of TEWL at that time point may be an average level of TEWL across that window of time. For example, the subject may be exposed to the suspected allergen (e.g. by ingesting the suspected allergen) and at at least one point following exposure TEWL may be measured.

In some embodiments, the methods of determining a treatment for a subject at risk of anaphylaxis comprise measuring a level of TEWL at multiple time points following exposure to the suspected allergen and determining the treatment for the subject based upon the levels of TEWL at those multiple time points. As described above, any suitable spacing between the first time point and the second time point may be used. For example, the first and second time points may be separated by a time span of less than 1 minute, about 1 minute, about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, or more than 15 minutes. For example, TEWL may be measured at multiple time points after exposure, such as multiple time points selected from immediately after exposure (i.e. less than 30 seconds after exposure), about 1 minute, about 2 minutes, about 3 minutes, about 4 minutes, about 5 minutes, about 6 minutes, about 7 minutes, about 8 minutes, about 9 minutes, about 10 minutes, or more than 10 minutes following exposure. For example, a level of TEWL may be measured immediately after exposure, a level of TEWL may be measured about 5 minutes after exposure, and level of TEWL may be measured about 10 minutes after exposure.

In some embodiments, measuring a level of transepidermal water loss (TEWL) in the subject following exposure to the suspected allergen comprises measuring a level of TEWL at a first time point following exposure to the suspected allergen, and measuring a level of TEWL in the subject at a second time point following exposure to the suspected allergen. In some embodiments, the second time point is after the first time point. In some embodiments, the first time point is no more than 4 hours (e.g. no more than 4 hours, no more than 3.5 hours, no more than 3 hours, no more than 2.5 hours, no more than 2 hours, no more than 1.5 hours, no more than 1 hour) after exposure to the suspected allergen, and the second time point is no more than 4 hours (e.g. no more than about 4 hours, no more than about 3.5 hours, no more than about 3 hours, no more than about 2.5 hours, no more than about 2 hours, no more than about 1.5 hours, no more than about 1 hour) after the first time point. In some embodiments, the first time point is no more than 2 hours after exposure to the suspected allergen, and the second time point no more than 2 hours after the first time point. In some embodiments, first time point is no more than 1 hour after exposure to the suspected allergen, and wherein the second time point is no more than 1 hour after the first time point. For example, in some embodiments the first time point is no more than 2 hours, no more than 1.5 hours, no more than 1 hour, no more than 50 minutes, no more than 45 minutes, no more than 40 minutes no more than 35 minutes, no more than 30 minutes, no more than 25 minutes, no more than 20 minutes, no more than 15 minutes, no more than 10 minutes, no more than 5 minutes, or about 1 minute after exposure to the suspected allergen and the second time point is no more than 2 hours, no more than 1.5 hours, no more than 1 hour, no more than 50 minutes, no more than 45 minutes, no more than 40 minutes no more than 35 minutes, no more than 30 minutes, no more than 25 minutes, no more than 20 minutes, no more than 15 minutes, no more than 10 minutes, no more than 5 minutes, or about 1 minute after the first time point.

In some embodiments, the treatment comprises an anti-anaphylactic agent when the level of TEWL at a time point following exposure to the suspected allergen is increased compared to a baseline level of TEWL in the subject; and/or the level of TEWL at the second time point following exposure to the suspected allergen is increased compared to a level of TEWL at the first time point following exposure to the suspected allergen. For example, in some embodiments the treatment comprises an anti-anaphylactic agent when the level of TEWL at a time point following exposure to the suspected allergen is increased compared to a baseline level of TEWL in the subject. As another example, in some embodiments, the treatment comprises an anti-anaphylactic agent when the level of TEWL at a second time point following exposure to the suspected allergen is increased compared to a level of TEWL at first time point following exposure to the suspected allergen.

In some embodiments, the treatment comprises an anti-anaphylactic agent when the level of TEWL following exposure to the suspected allergen is increased by at least an absolute amount compared to the baseline level. In some embodiments, the treatment comprises an anti-anaphylactic agent when the level of TEWL at a second time point following exposure to the suspected allergen is increased by at least an absolute amount compared to a level of TEWL at first time point following exposure to the suspected allergen. In some embodiments, the absolute amount is at least 0.5 g/m²/h, at least 0.6 g/m²/h, 0.7 g/m²/h, 0.8 g/m²/h, 0.9 g/m²/h, 1.0 g/m²/h, 1.1 g/m²/h, 1.2 g/m²/h, 1.3 g/m²/h, 1.4 g/m²/h, 1.5 g/m²/h, 1.6 g/m²/h, 1.7 g/m²/h, 1.8 g/m²/h, 1.9 g/m²/h, 2.0 g/m²/h, 2.1 g/m²/h, 2.2 g/m²/h, 2.3 g/m²/h, 2.4 g/m²/h, 2.5 g/m²/h, 2.6 g/m²/h, 2.7 g/m²/h, 2.8 g/m²/h, 2.9 g/m²/h, or at least 3.0 g/m²/h.

In some embodiments, the treatment comprises an anti-anaphylactic agent when the level of TEWL is at least doubled following exposure to the suspected allergen compared to the baseline level. In some embodiments, the treatment comprises an anti-anaphylactic agent when the level of TEWL at the second time point is at least double the level of TEWL at the first time point. In some embodiments, the treatment comprises an anti-anaphylactic agent when the level of TEWL is at least tripled following exposure to the suspected allergen compared to the baseline level. In some embodiments, the treatment comprises an anti-anaphylactic agent when the level of TEWL is at least tripled at the second time point compared to the level of TEWL at the first time point.

In some embodiments, the method further comprises determining whether one or more symptoms of allergy are present in the subject. For example, in some embodiments the method comprises determining whether one or more symptoms of allergy selected from hives, itching, sneezing, rhinorrhea, swelling, difficulty swallowing, shortness of breath, dizziness, nausea, and vomiting are present in the subject. In some embodiments, the treatment comprises an anti-anaphylactic agent when level of TEWL following exposure to the allergen is increased by at least 1 g/m²/hr compared to the baseline level and one or more symptoms of allergy are present in the subject. In some embodiments, the treatment comprises an anti-anaphylactic agent when the level of TEWL at the second time point is increased by at least 1 g/m²/hr compared to the level of TEWL at the first time point and one or more symptoms of allergy are present in the subject.

Suitable therapeutic agents include anti-anaphylactic agents, including epinephrine, bronchodilators (e.g. albuterol) and/or antihistamines (e.g. diphenhydramine, cetirizine, etc.). In some embodiments, the subject requires a medical intervention if anaphylaxis is predicted. In some embodiments, the medical intervention comprises providing an anti-anaphylactic agent to the subject. In some embodiments, the anti-anaphylactic agent is epinephrine. For example, epinephrine may be provided to the subject is anaphylaxis is predicted, thereby assisting in the resolution of an allergic reaction (e.g. anaphylaxis) and/or preventing severe anaphylaxis from occurring in the subject. The subject may also be monitored for anaphylaxis (e.g. severe anaphylaxis).

The methods described herein may assist in a determination of an oral food allergy in a subject. Accordingly, the methods described herein may be used in conjunction with an oral food challenge, and may be used to cease exposure and/or provide medical intervention to the suspected allergen prior to a potentially life-threatening anaphylactic response occurring in the subject. For example, a subject experiencing an increase in TEWL (e.g. an increase compared to baseline, an increase compared to an earlier time point, etc.) may be identified as at risk of experiencing anaphylaxis, and the food challenge may be ceased and/or medical intervention may be provided to the subject. The medical intervention may comprise providing a therapeutic agent to the subject and/or monitoring the subject. For example, the subject may be monitored for anaphylaxis. As another example, a therapeutic agent such as epinephrine may be provided to the subject. In some embodiments, epinephrine may be provided to the subject prior to a severe anaphylactic reaction in the subject (e.g. epinephrine may be provided preventatively). Accordingly, the methods descried herein facilitate accurate and safe oral food challenges to identify allergens in a subject without the usual risks associated with the oral food challenge itself. For example, in some embodiments provided herein is a method of identifying a food allergen in a subject. The method comprises providing the food allergen to the subject, and predicting anaphylaxis in the subject using a method described herein. In some embodiments, the methods further comprise providing an increased dose of the food allergen to the subject if anaphylaxis is not predicted. In some embodiments, the methods comprise providing serially increasing doses of the food allergen to the subject, and ceasing dosing of the allergen if anaphylaxis is predicted.

For any of the embodiments described herein, the subject may be a human. In some embodiments, the subject is an adult human subject (e.g. at least 18 years of age). In some embodiments, the subject is a pediatric human subject (e.g. between 1 year of age and 17 years of age). In some embodiments, the subject is an infant human subject (e.g. less than 1 year of age). The subject may be suspected of having a food allergy.

For any of the embodiments described herein, transepidermal water loss may be measured at any suitable location on the subject. For example, TEWL may be measured on the volar forearm, the clavicle, the neck, and/or the back (e.g. the upper back, the lower back) of the subject. In some embodiments, TEWL is measured at a single location (e.g. the volar forearm). In some embodiments, TEWL is measured at two locations (e.g. the volar forearm and the upper back of the subject). TEWL may be measured using any suitable device. In some embodiments, the device is a skin contact probe. For example, the device may be a skin contact probe placed gently upon the skin of the subject. A skin contact probe may be particularly useful for measuring a level of TEWL at a given point time. In some embodiments, the device is an adhesive probe. For example, the adhesive probe may be wirelessly connected to a computer interface (e.g. a monitor) which provides continuous TEWL measurements. In some embodiments, the device (e.g. the skin contact probe or the adhesive probe) comprises one or more sensors. For example, the device may comprise at least one temperature sensor and at least one humidity sensor. In some embodiments, the humidity sensor measures egressing water, thereby providing the measurement of TEWL in the subject.

### EXAMPLES

### Example 1

Background: Food allergy (FA) is a major societal health burden, affecting up to 10% of adults and 8% of children in the United States. FA causes food anaphylaxis, a life-threatening systemic allergic reaction causing 200,000 United States emergency room visits yearly. FA leads to childhood nutritional and growth deficiencies, debilitating patient and caregiver anxiety, and high costs related to epinephrine auto-injectors, healthcare visits, and food avoidance. FA diagnosis relies on history because current diagnostics including skin and blood food-specific immunoglobulin E (IgE) testing give positive predictive values as low as 50% and do not predict reaction severity. Therefore, an oral food challenge (OFC), the diagnostic standard for FA, should typically follow usual skin or blood FA testing to make a definitive diagnosis.

OFCs are risky and labor-intensive; during an OFC, the patient ingests the possibly allergic food in a graded fashion and may experience anaphylaxis or even death. During an OFC, the allergist and nurse conducting it monitor the patient for clinical changes in symptoms or the physical exam because there is no real-time monitoring device to definitively detect anaphylaxis. Furthermore, allergists face barriers to performing in-office OFCs, especially during the COVID-19 pandemic, limiting FA clinical practice and clinical trials. Adults are at the highest risk of fatal food anaphylaxis, which may further impact OFC accessibility for adults. However, performing OFCs gives key benefits, such as clarifying a diagnosis, allowing for an expanded diet, and improved quality-of-life. Patients who produce food-specific IgE but do not react on OFC are termed “food-sensitized”; those that react are “food-allergic”. Over-diagnosis via IgE testing without confirmatory OFCs leads to numerous adverse effects, which allergy leaders call a “disaster of misdiagnosis”. To improve diagnosis and care of FA patients during OFCs, an objective, accurate monitor of evolving anaphylaxis is needed to facilitate safer, more accessible OFCs.

Transepidermal water loss (TEWL) is a measure of skin barrier permeability. TEWL is the quantity of condensed water that diffuses across a fixed area of stratum corneum to the skin surface per unit time. The water evaporating from the skin is measured using a probe that is placed in contact with the skin surface and contains sensors that detect changes in water vapor density. TEWL is a sensitive measure that is affected by properties of the surrounding microclimate such as environmental humidity, temperature, and airflow. Accordingly, TEWL should be measured under controlled conditions.

Demonstrated herein are results that TEWL increases acutely before and during anaphylaxis in children. Further demonstrated herein is the use of continuous TEWL measurements in adult patients as a non-invasive, clinically applicable anaphylaxis monitoring modality during OFCs.

Results: TEWL is measured by temperature and humidity in a cross-sectional chamber on the skin (Alexander H, Brown S, Danby S, Flohr C. Research Techniques Made Simple: Transepidermal Water Loss Measurement as a Research Tool. J Invest Dermatol. November 2018; 138 (11): 2295-2300 el. doi: 10.1016/j.jid.2018.09.001). One static measurement takes 20 seconds. Measurements can be repeated in a non-invasive manner or can be taken continuously. TEWL is disturbed by variable airflow and room ventilation. OFCs were conducted in the same two rooms and TEWL devices were calibrated regularly, mitigating this issue.

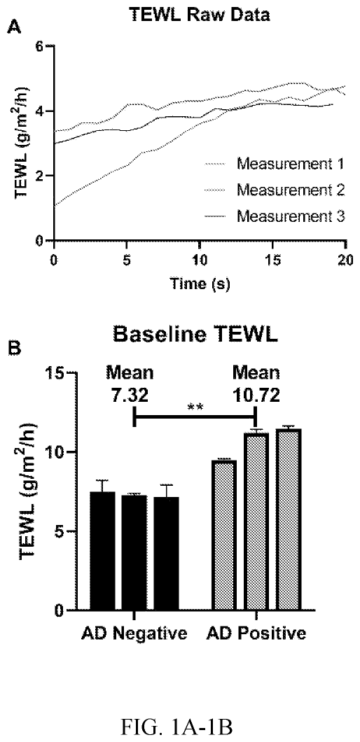

TEWL measurements were taken in triplicate from the volar forearm, clavicle, and neck using a Tewameter™ Hex device (Khazaka, Germany). FIG. 1A shows the time for equilibration of single TEWL measurements; measurement 1 shows how it takes at least 10 seconds for TEWL to stabilize after placement due to internal temperature changes from room temperature to skin temperature. Measurements 2 and 3 in FIG. 1A show stable measurements performed properly. FIG. 1B shows representative baseline TEWL values for 3 healthy individuals vs. 3 individuals with atopic dermatitis (AD). Intra-subject variability is minimal (FIG. 1B); AD subjects show an elevated baseline TEWL on normal skin. Together, these data show successful deployment and optimization of the use of tewameter devices, and that the measurement can give stable, repeatable baseline values from which to begin continuous monitoring.

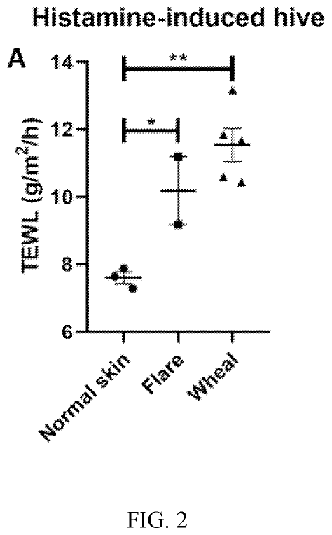

Anaphylaxis induces rapid blood vessel dilation with cutaneous heat and water loss. Facial or intranasal thermography can monitor anaphylaxis in real time, but due to various challenges such methods are not adopted in widespread use. Accordingly, histamine-induced skin hives were evaluated via TEWL. When a hive is induced on skin via histamine, a red flare arises followed by a wheal, a pale raised area. TEWL rises over both the flare (<1-2 minutes) and wheal (<10 minutes) of a hive (FIG. 2).

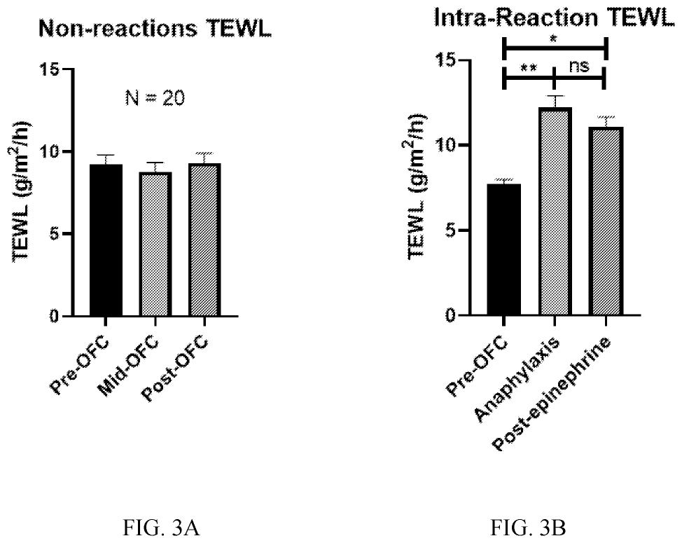

Additional patients were studied using methods described above. Results shown in FIG. 3A shows results from 20 patients. FIG. 3A shows mean TEWL values before the food challenge (pre-OFC), at the mid-point, and at the end for participants who did not react during a food challenge (i.e. non-reactors). FIG. 3B shows mean TEWL levels pre-PFC, at the mid-point (anaphylaxis) or post-epinephrine treatment for subjects who experienced an allergic response during the OFC. For this group, the OFC was conducted using peanuts as the allergen. Hives/vomiting occurred after dose 2-3, and TEWL rose before vomiting occurred. During a peanut-induced anaphylaxis episode with symptoms of hives and vomiting in an OFC patient, TEWL rose significantly from a baseline of 7.75 g/m²/h to 12.2 g/m²/h (FIG. 3B), even on skin without visible hives. There was a trend toward a decrease after epinephrine.

Accordingly, this data further exemplifies that TEWL levels provide an early indication of allergy in the subject, and that TEWL levels rise prior to onset of severe allergy symptoms. This data further supports the idea that continuous TEWL measurement during OFCs can provide a food anaphylaxis monitoring modality.

### Example 2

A double blood oral food challenge was conducted using 3 participants, 2 females and 1 male, ages 12-14. No patients had atopic dermatitis. Participants each experienced 3 placebo OFCs (i.e. non-allergens) and 6 reaction OFCs (milk, egg, peanut, cashew, and/or pistachio used as the allergen). Baseline TEWL, mid-OFC TEWL, and end-OFC levels were measured.

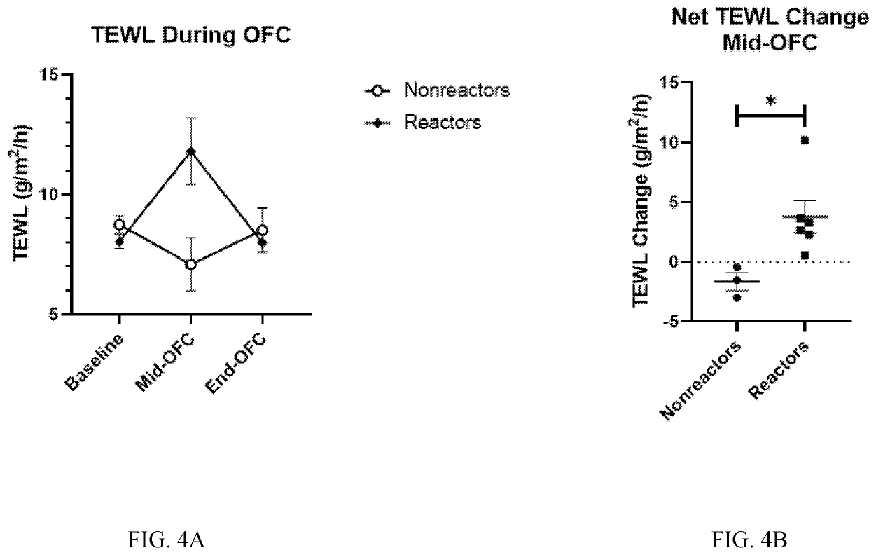

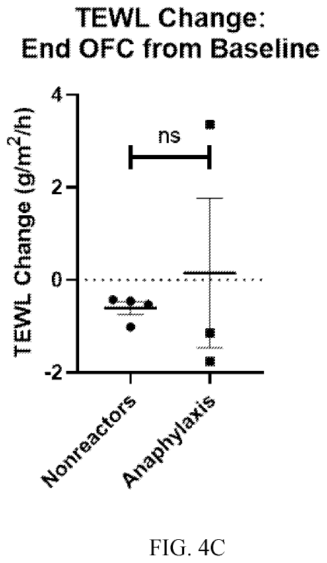

Next, the net change in TEWL during OFC-induced food anaphylaxis episodes was compared against TEWL measured during OFCs without a reaction. Across reactions, a significant increase in TEWL was seen at the time of anaphylaxis and prior to clinical anaphylaxis versus non-reactors. As shown in FIG. 4A, baseline TEWL was comparable for each participant. Reactors experienced a significant increase in the mean TEWL at the mid-OFC timepoint. Reactors were provided epinephrine, and symptoms were resolved prior to obtaining the end-OFC measurement. End-OFC TEWL levels were comparable for all participants. The mean net TEWL change for nonreactors vs. reactors at mid-OFC is shown in FIG. 4B. There was a significant (p<0.05) difference between reactors/nonreactors. No significant difference appears when comparing TEWL at the end of OFCs vs baseline among reactors and non-reactors (FIG. 4C, data shown for a subset of subjects) suggesting that epinephrine and resolution of the reaction leads to TEWL normalization.

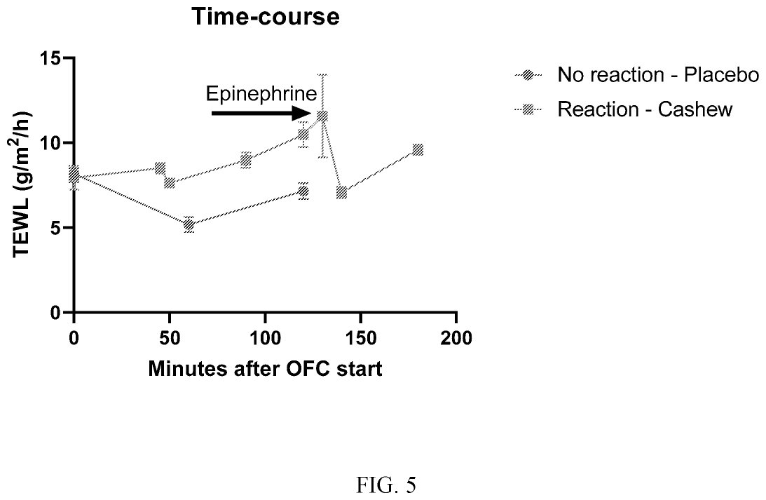

FIG. 5 shows the time course of TEWL changes for one patient who experienced anaphylaxis following cashew consumption. The figure illustrates an example of both non-reaction and reaction TEWL values as a time-course for the same person who had 2 challenges. In the placebo (blue) OFC with no reaction, TEWL drops slightly and then rises back to baseline. In the cashew reaction (red) OFC, TEWL begins to rise well before epinephrine is given (noted on figure). Accordingly, TEWL begins to rise well before the onset of clinical anaphylaxis in the subject. Note that the reaction challenge lasted longer because the subject was monitored until symptoms resolved, which takes longer than for a non-reacting challenge.

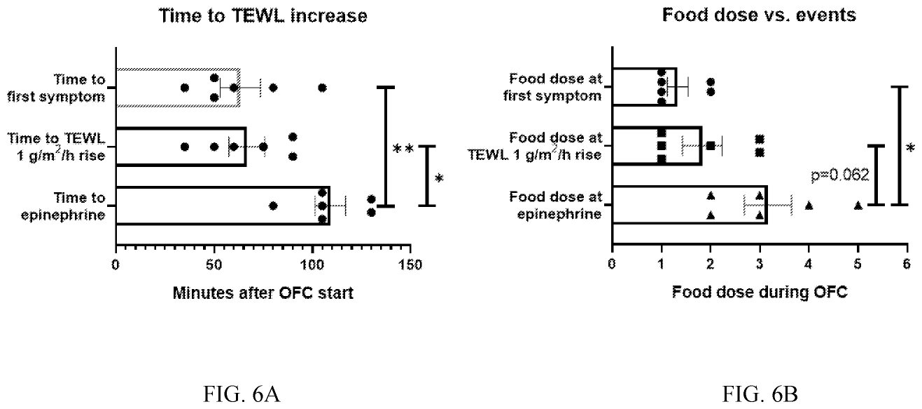

FIG. 6A shows that during challenges with reactions, the time during the OFC to first symptom is similar to the time it takes TEWL to rise 1 unit, and both of these are much shorter (by about 40 minutes) than the time to epinephrine (a definitive marker of clinical anaphylaxis). Accordingly, FIG. 6A demonstrates that TEWL increase in a subject can be used as a predictive marker for anaphylaxis, prior to clinical anaphylaxis occurring in the subject. A similar finding is shown in FIG. 6B, except this is delineated by food dose instead of time. Food challenges employ graduated doses given 15-20 minutes apart (minimum). FIG. 6B demonstrates that the food dose at the first symptom is similar to the food dose at TEWL rise by 1 unit. Importantly for both FIG. 6A and FIG. 6B, the first symptom and the rise in TEWL were both always before epinephrine (e.g. before clinical diagnosis of anaphylaxis), further evidencing that TEWL levels may be used to predict anaphylaxis in the subject prior to occurrence of clinical anaphylaxis, thus facilitating intervention prior to onset of more severe allergic reactions in the subject (e.g. severe anaphylaxis).

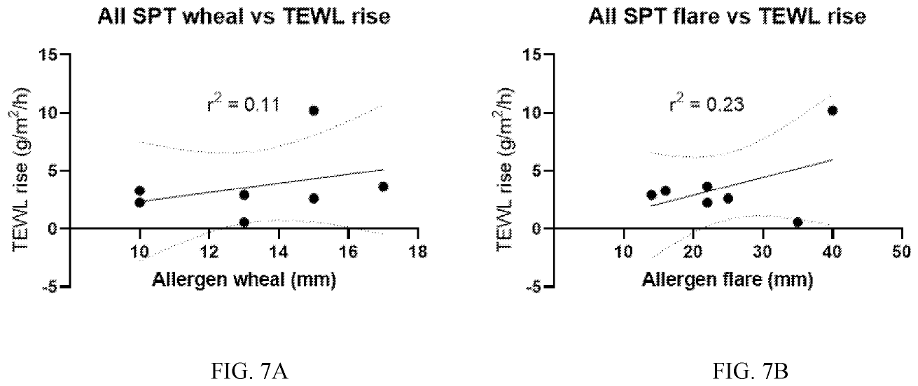

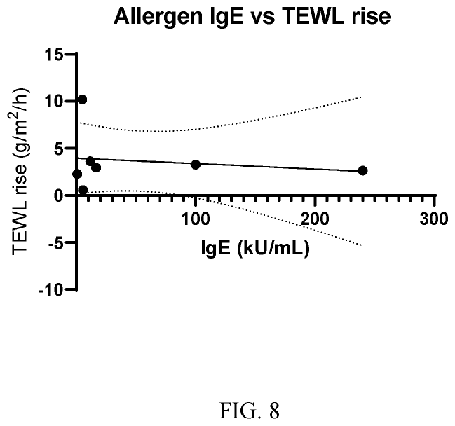

FIG. 7A-7B show that net TEWL rise during a reaction food challenge does not correlate well with the existing skin prick test on the wheal (L panel) or flare (R panel) for the same allergen. FIG. 8 shows that net TEWL rise during a reaction food challenge does not correlate well with Ige measurements for the subject following consumption of the same allergen. R² values are calculated using linear regression. Taken together, these data indicate that the methods of measuring TEWL in the subject described herein are an improvement over existing methods for measuring an allergic response in a subject.

### Example 3

TEWL may be applied as a real-time measurement of evolving anaphylaxis. Novel OFC stopping criteria based on TEWL can be defined. Accordingly, results from real-time TEWL measurements can be used to predict or detect anaphylaxis and allow challenges to be stopped prior to clinically evident anaphylaxis. By detecting anaphylaxis in real time, real-time TEWL measurements could help reduce the risk and cost of an OFC while preserving OFC accuracy as a criterion standard for FA.

Design: TEWL may be used to evaluate skin permeability in subjects prior to and during clinical care OFCs in adult (>age 18) biorepository enrollees. All OFC patients have food-specific skin and blood IgE testing done in addition to a thorough clinical FA history prior to OFCs. The patient may be without infections, asthma or AD exacerbation, or recent anaphylaxis. Patients may eat escalating food doses every 15 minutes up to a full serving, defined by food. Patients either react or do not during OFCs and may be observed for 1-2 hours after the last dose. All clinical data can be collected as a part of the described biorepository process in a REDCap database (Table 1). These stringent measures facilitate consistency in OFCs to allow reliable TEWL measurements.

TABLE 1
Example clinical data collected

| Baseline Data | OFC Data |
|---|---|
| Food allergies | Symptoms |
| Food allergy history | Anaphylaxis |
| Prior reaction details | Reaction severity |
| Food skin testing | Food dose/timing |
| Food IgE testing | Time of symptom |
| AD status | Epinephrine use |
| Asthma status | All medications |
| Rhinitis status | Medication timing |

Baseline and static TEWL measurement: Baseline measurements in triplicate on normal volar forearm, clavicle, and neck skin may be taken before the OFC for each subject. In a typical OFC, each patient ingests doses of food every 15-20 minutes, accordingly there is time to take TEWL measurements between each dose. Symptom timing in relation to TEWL measurements may be taken precisely to ensure that evolving reactions can be timed to specific TEWL changes. TEWL measurements may be obtained as soon as it is clinically appropriate as well as just prior to clinic discharge. If there is no reaction, TEWL measurements may be repeated after the final dose of food and just prior to clinic discharge.

Continuous TEWL measurement: All subjects may undergo continuous TEWL measurements. This can produce real-time results. Alternatively, results can be analyzed post-collection but are time-stamped with clinical data.

A Tewameter VT310 device (Khazaka, Germany) can be used for TEWL measurements. Measurements may be taken using a small adhesive to attach the probe to the skin. Measurements are commonly taken on the volar forearm. However, measurements on the upper back may be obtained as well. This would provide both a peripheral and a central measurement of TEWL and allow for a comparison of two body sites.

Data may be collected in MPA CT Plus software (Khazaka, Germany) which provides note-taking functions and exports data into Microsoft Excel (Seattle, WA) for analysis. All symptoms and medications may be timestamped onto TEWL data using this software. Static TEWL measurements between each food dose may also be taken to corroborate the performance of the continuous TEWL measure and to evaluate the contribution of noise to the measurement in this population.

Statistical Analysis: Various parameterizations for TEWL increases can be created. For example, suitable parameterizations that may be used include maximum TEWL, maximum increase in TEWL from baseline, doubling of TEWL from baseline (yes vs. no), tripling of TEWL from baseline, whether there was an increase by an absolute amount (e.g. 1 g/m²/h, +2, +3, etc.) from baseline, as well as assessing doubling/tripling/absolute increases from the prior minute instead of in reference from baseline.

In calculating these values, the continuous TEWL data can be censored among reactors at suitable time points prior to reaction (e.g. 2 minutes, 5 minutes, 15 minutes) to evaluate clinically meaningful lead times. Imposing a minimum sustained timespan meeting a set threshold (i.e., 30-60 seconds) may be explored to minimize noise effects in the measurement, essentially collapsing the time axis as an average TEWL per unit time (e.g. 30 seconds, 60 seconds, etc.). A series of logistic regression models testing the predictive value of various parameterizations can be employed. For example, logistic regression models testing the predictive value of various levels of absolute increases in TEWL or thresholds of TEWL and their combinations can be used. For such logistic regression models, two-thirds of the data may be used for the regression model and the remaining random third may be used for model validation. These models can be tested against occurrence, first symptom, anaphylaxis diagnosis, first medication use, and/or first epinephrine use as different outcomes. An optimal model may be selected based on strongest Area Under the Curve (AUC). The selected model may be applied to the validation sample and AUC, PPV, and NPV values may be reported based on different thresholds of predicted probability of response. These analyses will also explore confounding and subgroup variation including by age, sex, food ingested, asthma history, and AD.

### Example 4

Oral food challenges (OFCs) are the diagnostic standard for food allergy (FA), but are risky and labor-intensive. During OFCs, a patient ingests a possible food allergen and often experiences anaphylaxis. However, there are significant barriers to performing in-office OFCs for FA, limiting the use of this test in clinical practice and therapeutic development. One major barrier to more widespread utilization of OFCs is the risk of anaphylaxis from challenge. Given this, the development of an accurate, reliable anaphylaxis monitoring and/or prediction technique for OFCs is sorely needed. This example demonstrates that during OFCs, a TEWL rise anticipates a positive clinical challenge. TEWL presents a novel monitoring modality that may predict food anaphylaxis and facilitate improvements in OFC safety and tolerability.

### Methods

### Patient Population and Data Collection

This was an observational study in patients undergoing OFCs. All participants underwent either an open OFC or a double-blinded, placebo-controlled OFC for management of an existing or suspected food allergy. All participants or their parent/guardian(s) gave written informed consent for this study. Pediatric participants provided age-appropriate assent (assent was waived at age 6 and under).

### OFC Protocol

All OFCs were performed according to established methods. Briefly, participants had clinical food-specific skin and blood IgE testing in addition to a thorough clinical FA history prior to OFCs. Participants were well on the OFC date, without infections, asthma or atopic dermatitis exacerbations, or recent anaphylaxis. Participants ingested escalating food doses every 15-20 minutes up to a full serving for the food challenged. If no reaction occurred, participants were observed for at least 1 hour after the final food dose. If a reaction occurred, participants were assessed and treated by the attending allergist. Anaphylaxis was defined clinically by the attending allergist and treated with epinephrine, followed by other medications as clinically appropriate per the treating allergist. Anaphylaxis severity was graded using recorded symptoms from the OFC by an independent allergist not present during the OFC or treatment from the review of clinically documented symptoms according to CoFAR grading (Burks A W, Jones SM, Wood R A, Fleischer D M, Sicherer S H, Lindblad R W, et al. Oral immunotherapy for treatment of egg allergy in children. N Engl J Med. 2012; 367 (3): 233-43.). All clinical events, including food doses, symptoms, and treatments, were recorded and time-stamped by study staff.

### TEWL Measurement Methods

For all TEWL measurements, the skin areas utilized were required to be clean and dry. All measurement occurred in two climate-controlled rooms. Ambient temperature and humidity were measured. Participants were given time to acclimatize to the room before measurements were taken.

Discrete or “static” TEWL (STEWL) measurements were taken using the Tewameter Hex device (Courage+Khazaka gmbh, Cologne, Germany). Each measurement was taken in triplicate on the volar forearm (approximately ⅓ the distance from the wrist crease to the antecubital fossa, closer to the wrist), anterior lower neck above the clavicle, or on the upper back over the scapula. All measurements were taken on skin without visual evidence of rash, atopic dermatitis, hives, or other overt evidence of a dysfunctional skin barrier. After the location-finding portion of the study, all TEWL measurements were taken on the volar forearm only unless stated otherwise. When a reaction occurred, measurements were taken on visually normal, non-urticarial, non-flushed skin. Measurements during OFCs were taken just prior to ingesting the first dose of food, between each food dose, as soon as possible during a reaction (if possible, without interfering with clinical care), immediately after epinephrine, and at the end of the waiting period after a challenge (whether a reaction occurred or not).

Continuous TEWL (cTEWL) values were measured using the Tewameter VT310 (Courage+Khazaka gmbh, Cologne, Germany). The instrument was placed on the volar forearm using a 2-way adhesive. The instrument was in place prior to starting the OFC until the end but could be removed briefly as needed. These measurements were taken exclusively on skin without evidence of any barrier disruptions as well, as above.

All TEWL measurements were recorded using MPA Plus software (Courage+Khazaka gmbh, Cologne, Germany). Raw data were exported directly into Microsoft Excel (Microsoft, Seattle, WA) and analyzed as below (see Statistical Analysis). All TEWL data points were time stamped to be collated with the clinical OFC data, including times of food doses, symptoms, and treatments.

### Biomarker Analyses

All participants who consented to biosample collection provided a blood sample immediately prior to the OFC and another sample either at the end of the observation period (in the case of no reaction) or as soon as possible after a reaction was identified, without interfering with clinical care (in the case of a reaction). Samples were collected into K2 EDTA vacutainers and immediately placed in 4° C. and promptly separated via centrifuge to plasma. Plasma samples were aliquoted and immediately snap-frozen in liquid nitrogen until the time of analysis. Samples were not subjected to multiple freeze-thaw cycles.

The following analyses were performed using both pre- and post-OFC plasma samples. All kits were used according to manufacturer instructions: 1) ImmunoCAP® Tryptase fluoroenzymeimmunoassay analyzed on a Phadia™ 250 instrument (ThermoFisher Scientific, Uppsala, Sweden) was performed in the University of Michigan's CLIA-certified clinical laboratory; 2) Cytokines including tumor necrosis factor alpha (TNF-α), interleukins (IL) IL-1β, IL-3, IL-4, IL-5, IL-6, IL-10, IL-13, and vascular endothelial growth factor (VEGF) were analyzed via a customized cytokine/chemokine/growth factor immunology multiplex assay kit (Millipore/Milliplex).

### Statistical Analysis

All analyses were performed in Prism 8 (GraphPad Software Inc, San Diego, CA) and SAS software (SAS Institute, Cary, NC). Descriptive statistics were provided for both the static and continuous samples stratified by reactors vs. non-reactors using frequencies and percentages for categorical variables (chi-square for comparisons of reactors vs. non-reactors) and means, standard deviations, medians, interquartile ranges and ranges for continuous variables (and Kruskal-Wallis tests for comparisons). TEWL, and other inflammatory markers over time, were visualized using series, box and bar plots. Comparisons of reactors vs. non-reactors, where appropriate, were made with simple t-tests and ANOVAs. Linear regression models were also used to adjust for other characteristics such as age, sex, BMI, and history of asthma or AD.

Optimal thresholds for TEWL change in predicting reaction from continuous data were done by comparing maximum TEWL values before and after the first dose. We explored the predicting value of 1 or 2 g TEWL rises in predicting subsequent reaction, and the change in predictive value when also requiring the presence of a reported symptom.

### Results

Table 2 details the composition of the sTEWL measurement cohort. sTEWL measurements were obtained from 125 OFCs. The mean age was 8.7 years. The group was 38% female, predominantly White, and included a high proportion of individuals with other atopic conditions, including atopic dermatitis (AD), asthma, and allergic rhinitis/rhinoconjunctivitis. A broad array of food allergies was represented, particularly milk, egg, peanut, tree nut, and sesame allergies. Baseline demographic characteristics were largely similar between groups.

TABLE 2
Cohort Details

| | Overall | | Reacted | | No reaction | | p-value |
|---|---|---|---|---|---|---|---|
| Age (years) | | | | | | | 0.09 |
| Mean (SD) | 8.7 | (6.68) | 10.5 | (4.89) | 8.5 | (6.90) | |
| Median (IQR) | 7.0 (3.0 to | | 11.0 (8.0 to | | 6.0 (3.0 to | | |
| | 13.0) | | 14.0) | | 12.0) | | |
| Range | 1.0 to 35.0 | | 2.0 to 18.0 | | 1.0 to 35.0 | | |
| N | 125 | | 17 | | 108 | | |
| N missing | 0 | | 0 | | 0 | | |
| Sex, n (%) | | | | | | | 0.18 |
| Male | 77 | (62) | 8 | (47) | 69 | (64) | |
| Female | 48 | (38) | 9 | (53) | 39 | (36) | |
| Race | | | | | | | 0.76 |
| White | 90 | (72) | 13 | (76) | 77 | (71) | |
| Black or African American | 8 | (6) | 1 | (6) | 7 | (6) | |
| Asian, American Indian, or Alaska | 11 | (9) | 2 | (12) | 9 | (8) | |
| Native | | | | | | | |
| Other | 3 | (2) | 0 | (0) | 3 | (3) | |
| More than one race | 4 | (3) | 1 | (6) | 3 | (3) | |
| Missing | 9 | (7) | 0 | (0) | 9 | (8) | |
| Hispanic Ethnicity, n (%) | 7 | (6) | 0 | (0) | 7 | (6) | 0.47 |
| BMI | | | | | | | 0.86 |
| Mean (SD) | 19.7 | (5.81) | 19.8 | (5.19) | 19.7 | (5.93) | |
| Median IQR) | 18.1 (16.3 | | 17.2 (16.3 to | | 18.1 (16.3 to | | |
| | to 22.3) | | 24.3) | | 22.0) | | |
| Range | 0.0 to 47.6 | | 14.5 to 32.1 | | 0.0 to 47.6 | | |
| N | 125 | | 17 | | 108 | | |
| N missing | 0 | | 0 | | 0 | | |
| Food allergies, n (%) | | | | | | | |
| Chickpea | 8 | (6) | 2 | (12) | 6 | (6) | 0.33 |
| Milk | 41 | (33) | 6 | (35) | 35 | (32) | 0.81 |
| Other grains | 3 | (2) | 0 | (0) | 3 | (3) | 0.49 |
| Fruits | 4 | (3) | 0 | (0) | 4 | (4) | 0.42 |
| Vegetables | 3 | (2) | 1 | (6) | 2 | (2) | 0.31 |
| Legumes | 7 | (6) | 1 | (6) | 6 | (6) | 0.96 |
| Meats | 2 | (2) | 1 | (6) | 1 | (1) | 0.13 |
| Egg | 59 | (47) | 7 | (41) | 52 | (48) | 0.59 |
| Wheat | 5 | (4) | 1 | (6) | 4 | (4) | 0.67 |
| Soy | 10 | (8) | 0 | (0) | 10 | (9) | 0.19 |
| Peanut | 75 | (60) | 13 | (76) | 62 | (57) | 0.13 |
| Tree Nuts | 64 | (51) | 8 | (47) | 56 | (52) | 0.71 |
| Fish | 1 | (1) | 0 | (0) | 1 | (1) | 0.69 |
| Shellfish | 7 | (6) | 1 | (6) | 6 | (6) | 0.96 |
| Sesame | 19 | (15) | 0 | (0) | 19 | (18) | 0.06 |
| General allergy history, n (%) | | | | | | | |
| Atopic dermatitis | 82 | (66) | 7 | (41) | 75 | (69) | 0.02 |
| Asthma | 49 | (39) | 6 | (35) | 43 | (40) | 0.86 |
| Allergic rhinitis/rhinoconjunctivitis | 64 | (51) | 10 | (59) | 54 | (50) | 0.50 |
| Wheal | | | | | | | <0.001 |
| Mean (SD) | 5.5 | (4.58) | 10.8 | (5.36) | 4.4 | (3.56) | |
| Median IQR) | 5.0 (2.0 to | | 10.0 (7.0 to | | 4.0 (2.0 to | | |
| | 8.0) | | 14.0) | | 7.0) | | |
| Range | 0.0 to 23.0 | | 2.0 to 23.0 | | 0.0 to 14.0 | | |
| N | 93 | | 16 | | 77 | | |
| N missing | 32 | | 1 | | 31 | | |
| Flare | | | | | | | 0.01 |
| Mean (SD) | 17.1 | (15.73) | 24.4 | (14.34) | 15.6 | (15.67) | |
| Median IQR) | 15.5 (4.0 | | 22.0 (15.0 to | | 15.0 (2.5 to | | |
| | to 24.5) | | 33.5) | | 22.0) | | |
| Range | 0.0 to 85.0 | | 3.0 to 60.0 | | 0.0 to 85.0 | | |
| N | 92 | | 16 | | 76 | | |
| N missing | 33 | | 1 | | 32 | | |
| sIgE | | | | | | | 0.004 |
| Mean (SD) | 3.2 | (7.79) | 5.3 | (5.14) | 2.8 | (8.15) | |

| | | | |
|---|---|---|---|
| Median IQR) | 0.6 (0.0 to | 5.3 (0.6 to | 0.6 (0.0 to |
| | 2.7) | 8.4) | 1.5) |
| Range | 0.0 to 52.8 | 0.0 to 16.5 | 0.0 to 52.8 |
| N | 92 | 14 | 78 |
| N missing | 33 | 3 | 30 |

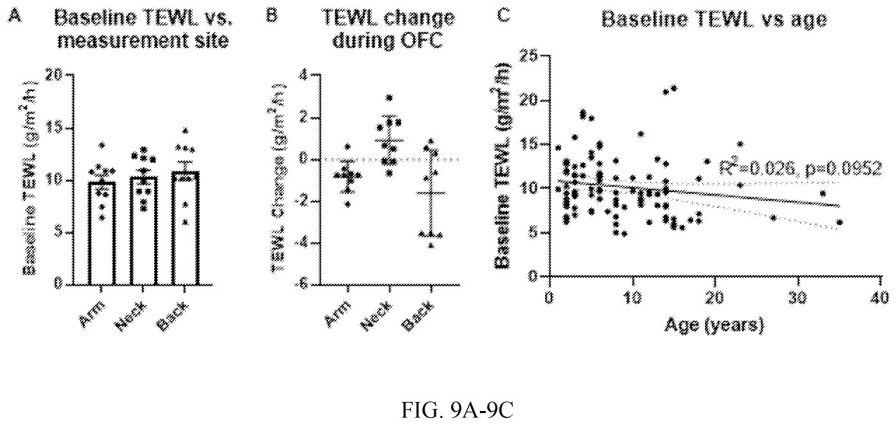

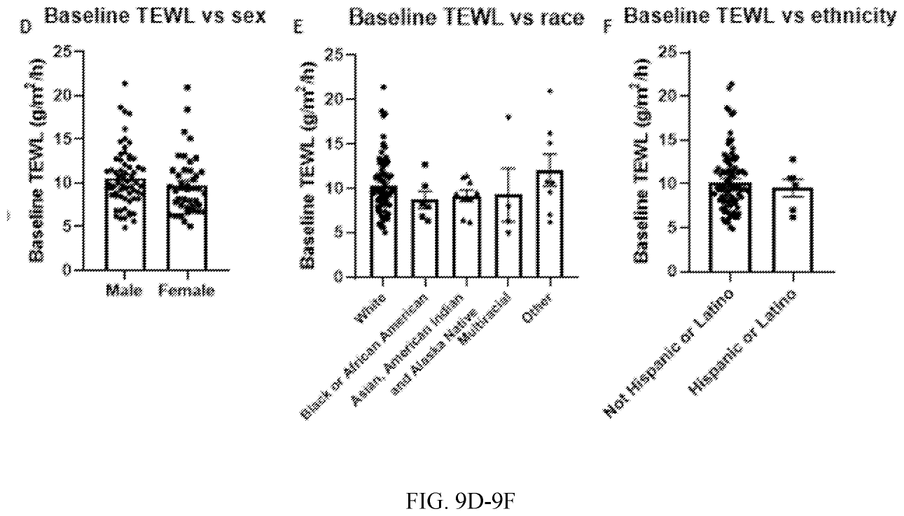

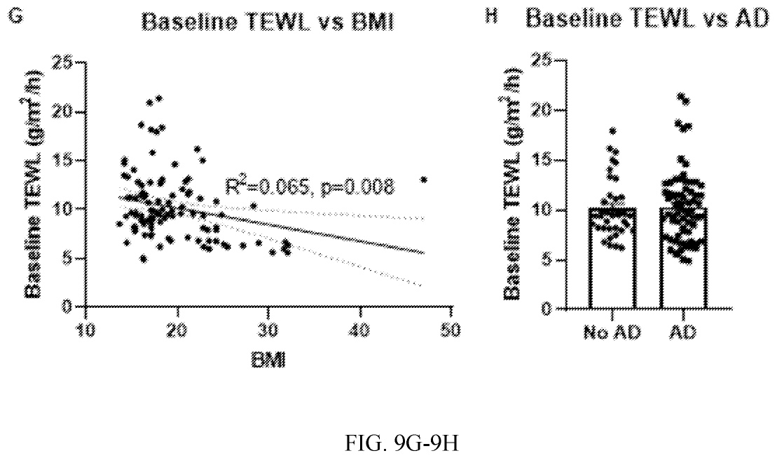

In initial studies the location of the TEWL measurement was optimized to determine the most consistent method of data collection. Baseline TEWL measurements were compared by body site in the first 10 non-reactive participants and found no significant difference between sites at baseline (FIG. 9A). The most consistent measurements were from the forearm where measurements were stable and changed little, whereas measurements on the neck or back showed substantial variability and a notable change from baseline (FIG. 9B). Given the ease of measuring TEWL on the forearm and these favorable measurement characteristics, the rest of the study was conducted using only forearm measurements. Using forearm measurement, baseline TEWL decreased non-significantly with increasing age (FIG. 9C) but did not vary significantly by sex (FIG. 9D), race (FIG. 9E), or ethnicity (FIG. 9F). A small decrease in TEWL was associated with greater BMI (FIG. 9G) and there was a trend toward a greater baseline TEWL among participants with AD, but neither were significant (FIG. 9H). Thus, the forearm allowed consistent baseline data accumulation that can be used to compare reacting vs. non-reacting patient populations.

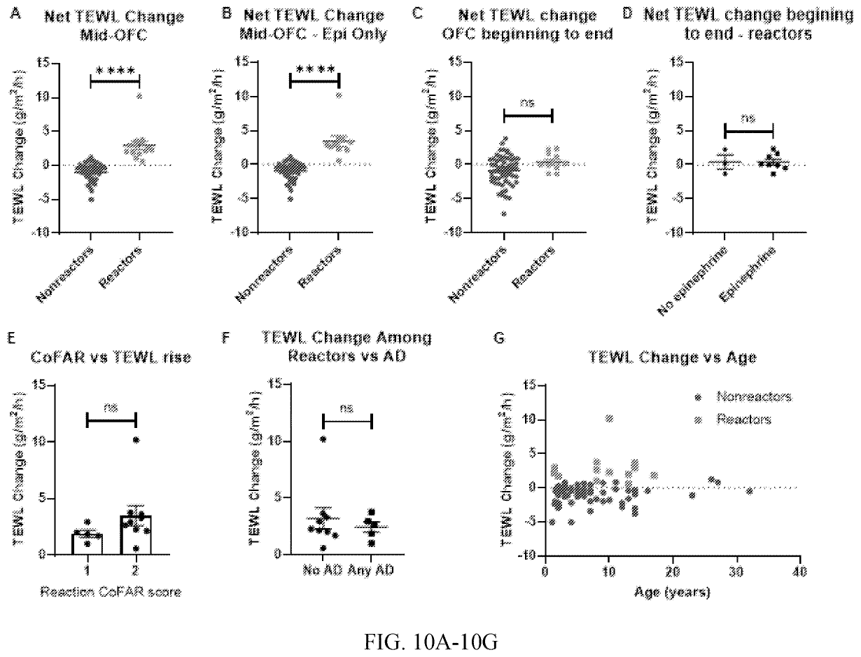

The change in TEWL obtained before the OFC started (baseline) was compared with the value from the midpoint of the OFC (generally after food dose 3) for non-reactors and to the last measurement before anaphylaxis for reactors. TEWL rose significantly during OFC reactions but was largely unchanged in the absence of a reaction (FIG. 10A). The rise in TEWL was also present when only severe reactions that required epinephrine were compared to non-reactors (FIG. 10B). TEWL values in patients with allergic reactions returned to similar levels as the unchanged non-reactors by the end of the challenge regardless of whether reactors received epinephrine (FIG. 10C, D). There was a trend toward a greater TEWL rise during CoFAR grade 2 reactions versus grade 1 reactions, but this was not significant (FIG. 10E), and no reaction of grade 3 or higher occurred during the study period. The rise in TEWL noted during reactions was similar regardless of whether the subject had AD (FIG. 10F) and across the age spectrum (FIG. 10G).

Given the TEWL changes documented here occurred on uninvolved skin (FIG. 10A, 11B), it was evaluated whether TEWL increases over the wheal or flare of urticarial lesions. TEWL increases over both the wheal and the flare of histamine-induced hives as compared to normal skin. Whether the increase in TEWL seen correlated with baseline food skin or blood IgE testing was also evaluated. The degree of food sensitization defined by skin test wheal, skin test flare, or food-specific IgE did not show a significant correlation with the degree of TEWL change during OFC. Together, these data indicate that TEWL measurements correlate with the induction of a systemic anaphylactic response to OFC.

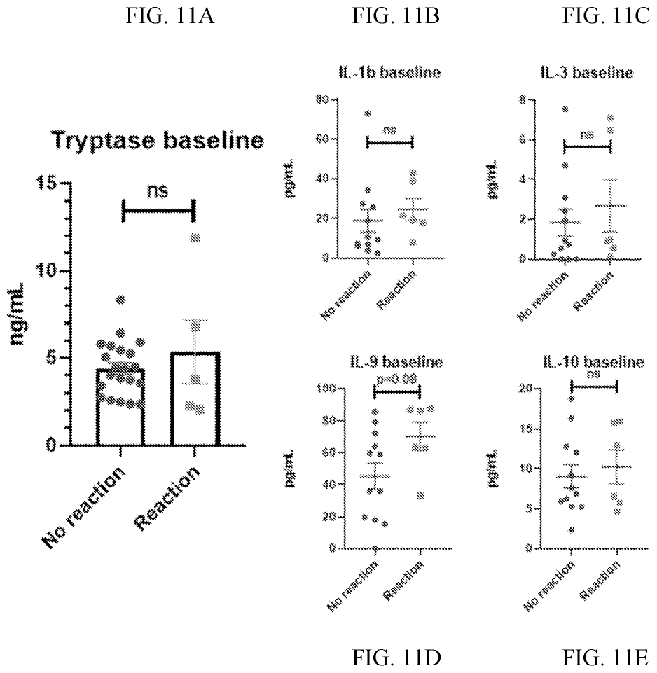

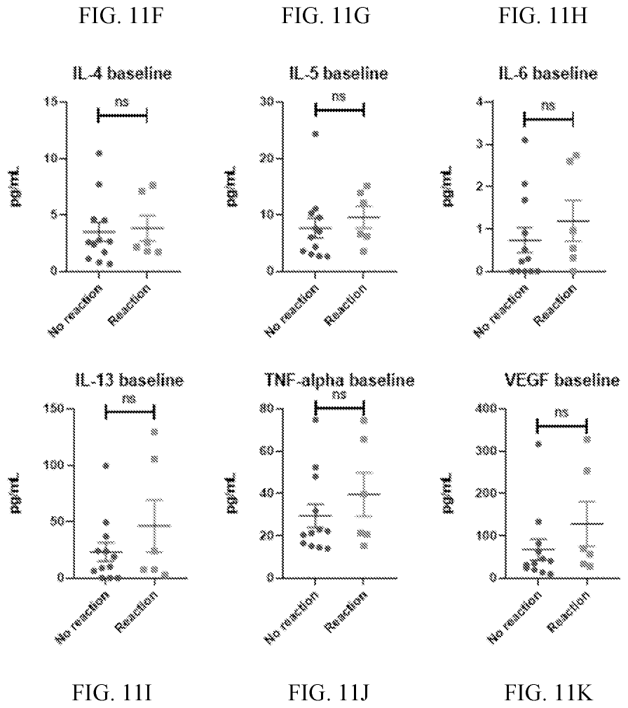

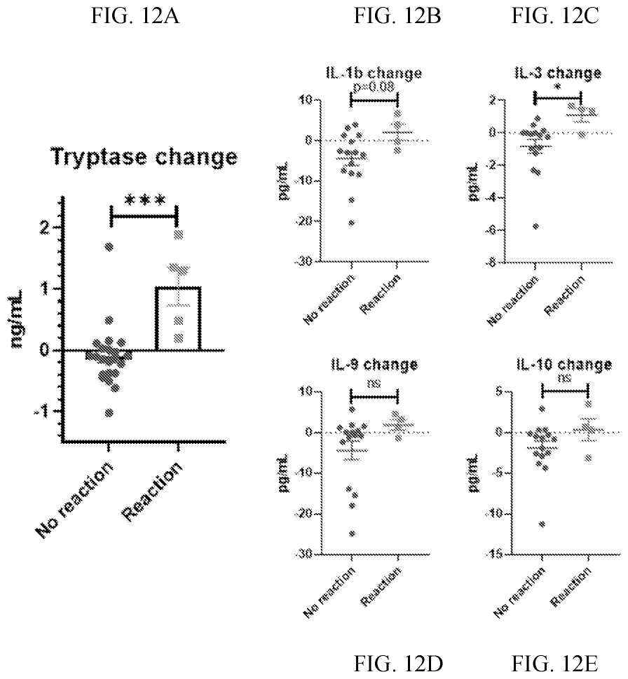

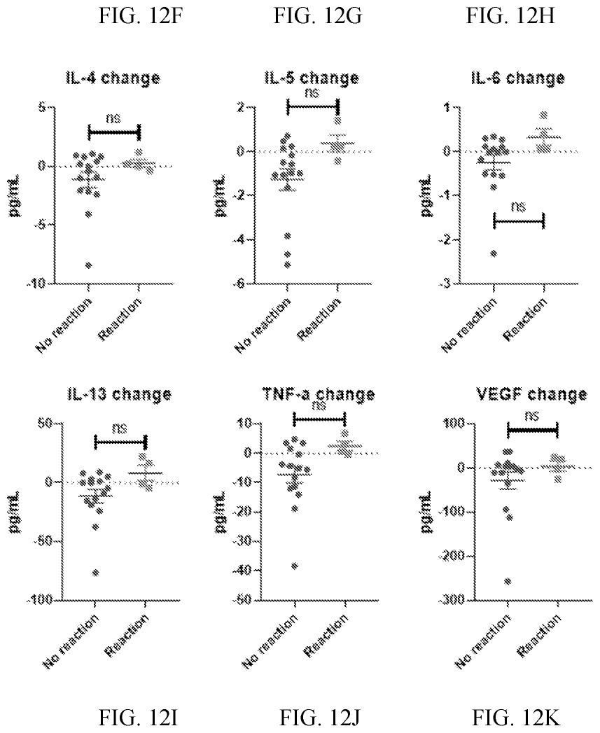

It was next investigated whether the clinical reactions observed correlated with biochemical evidence of anaphylaxis. Plasma from all participants that consented to give blood was analyzed. Samples were screened for immune markers including tryptase, TNF-α, interleukins (IL) IL-1β, IL-3, IL-4, IL-5, IL-6, IL-9, IL-10, IL-13, and VEGF. At baseline, there were no significant differences between the reactor group and the non-reactor group in any marker tested (FIG. 11A-K). A trend (p=0.09) toward a greater IL-9 systemic level was seen among reactors, which may reflect prior studies indicating a role for elevated IL-9 activity in food allergic individuals (31-33). In contrast, a significant increase in tryptase and IL-3 results was seen compared to baseline in subjects during clinical allergic reactions (FIG. 12). There were also trends toward increases in IL-1β, IL-6, and TNF-α in the subjects with reactions, but no significant change in VEGF.

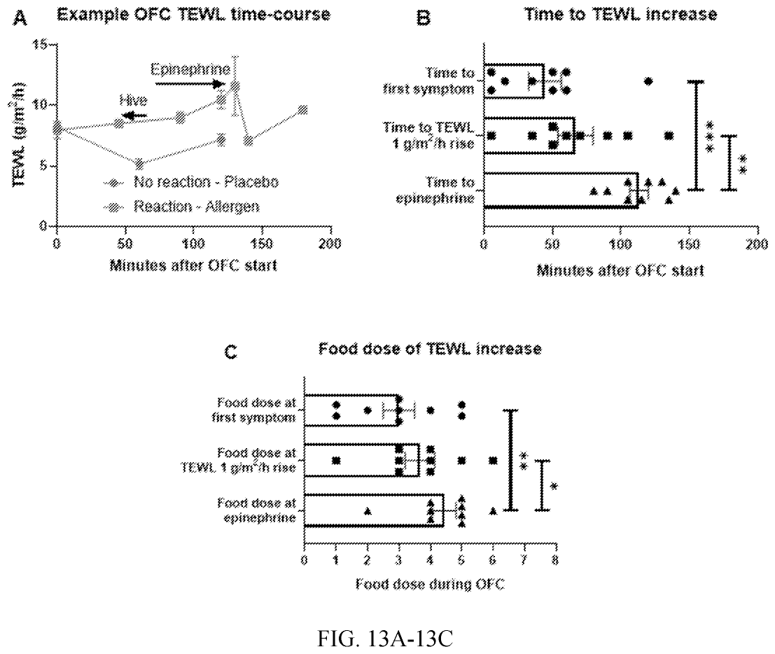

The timing of the increase in TEWL observed among reactors as in FIGS. 1A and 1B was next investigated. The timing of TEWL values for reactors during OFCs was assessed. Most reactions demonstrated a steady rise in TEWL from baseline, followed by a decrease after epinephrine (FIG. 13A). The time to first symptom, the time to a TEWL rise of 1 g/m²/h from baseline, and the time to epinephrine administration was quantified according to minutes after the start of the OFC and according to the food dose during which an event occurred. The time to a first symptom and the time to a 1-unit TEWL rise were each significantly less than the time to epinephrine (FIG. 13B). Similarly, the food dose on which the first symptom or 1-unit TEWL rise occurred was substantially earlier than the food dose of epinephrine administration (FIG. 13C).

Table 3 defines the baseline characteristics of the individuals who had cTEWL measurement during the OFCs. The mean age was 13.5 years, older than the sTEWL group. The group was 40% female, also predominantly White, and again included a large proportion of individuals with other atopic conditions. Again, a broad array of food allergies was represented. Baseline demographic characteristics were largely similar between the reactor and non-reactor groups statistically.

TABLE 3
Baseline Characteristics of Individuals who had cTEWL Measurements during OFC

| | Overall | | Reacted | | No reaction | | p-value |
|---|---|---|---|---|---|---|---|
| Age (years) | | | | | | | 0.34 |
| Mean (SD) | 13.5 | (10.39) | 9.7 | (5.92) | 13.8 | (10.62) | |
| Median IQR) | 11.0 (5.0 to | | 9.0 (5.0 to | | 11.5 (5.0 to | | |
| | 18.5) | | 16.0) | | 19.0) | | |
| Range | 2.0 to 58.0 | | 2.0 to 17.0 | | 3.0 to 58.0 | | |
| N | 84 | | 6 | | 78 | | |
| N missing | 0 | | 0 | | 0 | | |
| Sex, n (%) | | | | | | | 0.71 |
| Male | 50 | (60) | 4 | (67) | 46 | (59) | |
| Female | 34 | (40) | 2 | (33) | 32 | (41) | |
| Race | | | | | | | 0.04 |
| White | 62 | (74) | 3 | (50) | 59 | (76) | |
| Black or African American | 5 | (6) | 2 | (33) | 3 | (4) | |
| Asian, American Indian and Alaska | 5 | (6) | 0 | (0) | 5 | (6) | |
| Native | | | | | | | |
| Other | 4 | (5) | 0 | (0) | 4 | (5) | |
| More than one race | 4 | (5) | 1 | (17) | 3 | (4) | |
| Missing | 4 | (5) | 0 | (0) | 4 | (5) | |
| Hispanic Ethnicity, n (%) | 4 | (5) | 0 | (0) | 4 | (5) | 0.82 |
| BMI | | | | | | | 0.70 |
| Mean (SD) | 21.1 | (7.21) | 20.2 | (5.96) | 21.2 | (7.32) | |
| Median IQR) | 19.2 (16.9 to | | 18.1 (16.7 to | | 19.8 (16.9 to | | |
| | 23.5) | | 19.8) | | 23.6) | | |
| Range | 0.0 to 47.6 | | 16.4 to 32.1 | | 0.0 to 47.6 | | |
| N | 84 | | 6 | | 78 | | |
| N missing | 0 | | 0 | | 0 | | |
| Food allergies, n (%) | | | | | | | |
| Chickpea | 5 | (6) | 0 | (0) | 5 | (6) | 0.52 |
| Milk | 21 | (25) | 2 | (33) | 19 | (24) | 0.62 |
| Other grains | 2 | (2) | 0 | (0) | 2 | (3) | 0.69 |
| Fruits | 4 | (5) | 0 | (0) | 4 | (5) | 0.57 |
| Vegetables | 3 | (4) | 0 | (0) | 3 | (4) | 0.62 |
| Legumes | 8 | (10) | 0 | (0) | 8 | (10) | 0.41 |
| Meats | 1 | (1) | 1 | (17) | 0 | (0) | <0.001 |
| Egg | 36 | (43) | 3 | (50) | 33 | (42) | 0.71 |
| Wheat | 3 | (4) | 1 | (17) | 2 | (3) | 0.07 |
| Soy | 6 | (7) | 1 | (17) | 5 | (6) | 0.35 |
| Peanut | 47 | (56) | 3 | (50) | 44 | (56) | 0.76 |
| Tree Nuts | 57 | (68) | 4 | (67) | 53 | (68) | 0.95 |
| Fish | 5 | (6) | 0 | (0) | 5 | (6) | 0.52 |
| Shellfish | 8 | (10) | 1 | (17) | 7 | (9) | 0.54 |
| Sesame | 15 | (18) | 1 | (17) | 14 | (18) | 0.94 |
| General allergy history, n (%) | | | | | | | |
| Atopic dermatitis | 55 | (65) | 4 | (67) | 51 | (65) | 0.001 |
| Asthma | 35 | (42) | 4 | (67) | 31 | (40) | 0.43 |
| Allergic rhinitis/rhinoconjunctivitis | 55 | (65) | 3 | (50) | 52 | (67) | 0.65 |
| Wheal | | | | | | | 0.16 |
| Mean (SD) | 4.1 | (2.96) | 6.0 | (3.35) | 3.9 | (2.88) | |
| Median IQR) | 4.0 (2.0 to | | 6.5 (3.0 to | | 4.0 (2.0 to | | |
| | 5.5) | | 9.0) | | 5.0) | | |
| Range | 0.0 to 12.0 | | 2.0 to 9.0 | | 0.0 to 12.0 | | |
| N | 60 | | 6 | | 54 | | |
| N missing | 24 | | 0 | | 24 | | |
| Flare | | | | | | | 0.32 |
| Mean (SD) | 14.3 | (12.36) | 17.8 | (10.70) | 13.9 | (12.56) | |
| Median IQR) | 11.5 (4.0 to | | 20.0 (8.0 to | | 10.0 (4.0 to | | |
| | 23.0) | | 24.0) | | 22.0) | | |
| Range | 0.0 to 50.0 | | 3.0 to 32.0 | | 0.0 to 50.0 | | |
| N | 60 | | 6 | | 54 | | |
| N missing | 24 | | 0 | | 24 | | |
| sIgE | | | | | | | 0.60 |
| Mean (SD) | 3.7 | (9.11) | 2.2 | (2.42) | 3.8 | (9.58) | |

| | | | |
|---|---|---|---|
| Median IQR) | 0.9 (0.0 to | 1.6 (0.5 to | 0.9 (0.0 to |
| | 2.5) | 3.4) | 1.6) |
| Range | 0.0 to 52.8 | 0.0 to 6.4 | 0.0 to 52.8 |
| N | 59 | 6 | 53 |
| N missing | 25 | 0 | 25 |

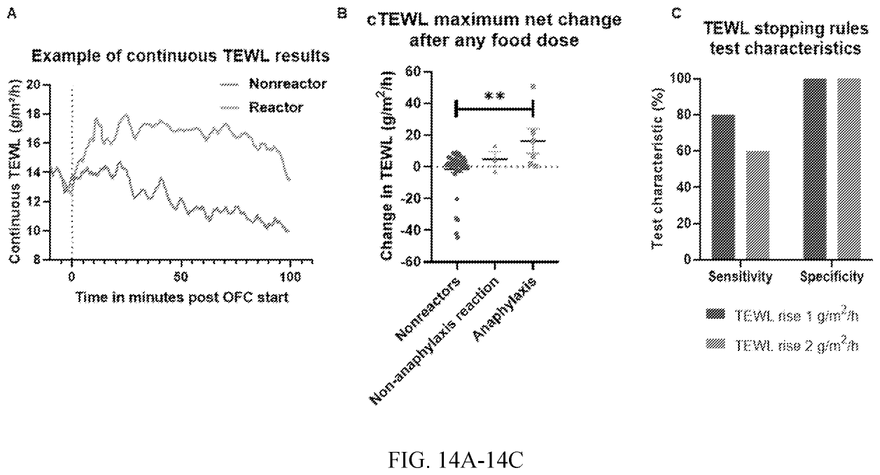

Given the favorable findings from the static TEWL measurements, continuous TEWL monitoring was evaluated in a subsequent group of OFCs. Typical results for subjects with and without allergic reactions are shown (FIG. 14A). When taken together the maximal net TEWL change after any food dose was significantly greater in the subjects with anaphylaxis than in either those having no reaction group or those having reactions without clinical anaphylaxis (FIG. 14B).

Based on these results, a TEWL change that would predict anaphylaxis before it was clinically evident was defined. The onset of frank anaphylaxis was defined as the time of epinephrine administration, a variety of potential OFC stopping rules were tested using these data. It was found that a 1 g/m²/h rise in TEWL plus any single symptom or objective sign of an allergic reaction (such as a hive, mouth itching, or rhinorrhea), produced 80% sensitivity and 100% specificity for impending anaphylaxis and provided an average of 38 minutes of warning prior to clinical anaphylaxis (FIG. 14C).

### DISCUSSION

This example demonstrates that TEWL can be readily measured during OFCs and significantly increases during food allergic reactions and anaphylaxis. In addition, this rise correlates with biochemical markers of anaphylaxis and substantially precedes clinical detection of anaphylaxis. Using a monitoring-capable version of a commercial TEWL measurement device, it was shown herein that TEWL monitoring presents a viable predictor of impending anaphylaxis well in advance of clinical reaction. Together, these observations demonstrate the use of TEWL as an anaphylaxis prediction method in OFC.

Given the high prevalence of FA worldwide and the adverse effects associated with an inaccurate FA diagnosis, correct FA diagnoses are critical. The OFC remains central to diagnosis due to high false positive rates for skin and blood food testing. The of TEWL changes observed in participants during this study correlated only with clinical reactivity and not with food-specific skin or blood IgE testing, suggesting TEWL measurement reflects clinical reactivity and not sensitization.

Barriers to performing OFCs take multiple forms, and there is widespread interest in making this test safer and more accessible. Prior attempts to use other methods to detect anaphylaxis in real time have not entered widespread use. For example, facial thermography requires optics expertise, a specific, high-quality camera, tightly controlled ambient conditions (including temperature and cold lighting), and extremely compliant subjects willing to sit at a fixed distance from a camera for repeated takes. Given that many OFCs for FA are performed in children, the potential limitations associated with participant behavioral non-adherence alone are obvious. In contrast, TEWL is a simple measurement that can be readily replicated in offices without complex equipment. TEWL can also be affixed to the skin and provide meaningful data even among children, as demonstrated herein. Accordingly, TEWL provides a valuable enhancement of OFCs for clinical use, since the ability to detect and predict anaphylaxis prior to the need for epinephrine would improve the safety of this test.

In sum, the data presented herein demonstrate that during OFCs, a TEWL rise anticipates a positive clinical challenge. Therefore, TEWL presents a novel monitoring modality that predicts food anaphylaxis and facilitates improvements in OFC safety and tolerability.

It is understood that the foregoing detailed description and accompanying examples are merely illustrative and are not to be taken as limitations upon the scope of the disclosure, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the disclosure, may be made without departing from the spirit and scope thereof.

Any patents and publications referenced herein are herein incorporated by reference in their entireties.

1-111. (canceled)

112. A method of treating allergen-induced anaphylaxis in a subject, the method comprising:
a) exposing the subject to a suspected allergen;
b) measuring a level of transepidermal water loss (TEWL) in the subject following exposure to the suspected allergen to determine risk of anaphylaxis in the subject; and
c) administering an anti-anaphylactic agent to the subject determined to be at risk for anaphylaxis.

113. The method of claim 112, wherein measuring a level of TEWL in the subject following exposure to the suspected allergen comprises continuously measuring TEWL in the subject for a duration of time.

114. The method of claim 113, wherein the duration of time is 15 seconds to 6 hours, 15 seconds to 4 hours, or 15 seconds to 2 hours.

115. The method of claim 112, wherein measuring a level of TEWL in the subject following exposure to the allergen comprises measuring a level of TEWL at at least one time point following exposure to the suspected allergen, wherein the time point is no more than 4 hours after exposure to the suspected allergen.

116. The method of claim 115, wherein the time point is no more than 2 hours after exposure to the suspected allergen.

117. The method of claim 112, wherein an increase in the level of TEWL at a time point following exposure to the suspected allergen compared to a baseline level of TEWL in the subject indicates the subject as at risk for anaphylaxis, wherein the time point is no more than 4 hours after exposure to the suspected allergen.

118. The method of claim 117, wherein the baseline level of TEWL is obtained from the subject no more than 4 hours before exposure to the suspected allergen.

119. The method of claim 118, wherein the baseline level of TEWL is obtained from the subject no more than 1 hour before exposure to the suspected allergen.

120. The method of claim 117, wherein an increase in the level of TEWL by at least an absolute amount compared to the baseline level indicates the subject as at risk for anaphylaxis.

121. The method of claim 120, wherein the absolute amount is 1 g/m²/hr, 2 g/m²/hr, or 3 g/m²/hr.

122. The method of claim 117, wherein a level of TEWL that is at least doubled compared to the baseline level indicates the subject as at risk for anaphylaxis.

123. The method of claim 112, wherein an increase in the level of TEWL at a second time point following exposure to the suspected allergen compared to a level of TEWL at first time point following exposure to the suspected allergen indicates the subject as at risk for anaphylaxis, wherein the first time point is no more than 4 hours after exposure to the suspected allergen and the second time point is no more than 4 hours after the first time point.

124. The method of claim 123, wherein an increase in the level of TEWL at the second time point by at least an absolute compared to the first time point indicates the subject as at risk for anaphylaxis.

125. The method of claim 124, wherein the absolute amount is 1 g/m²/hr, 2 g/m²/hr, or 3 g/m²/hr.

126. The method of claim 123, wherein a level of TEWL at the second time point that is at least doubled compared to the level at the first time point indicates the subject as at risk for anaphylaxis.

127. The method of claim 112, wherein the suspected allergen is a suspected food allergen.

128. The method of claim 127, wherein the subject is exposed to the suspected food allergen by oral consumption of the suspected food allergen.

129. The method of claim 128, wherein the subject is a human.

130. The method of claim 129, wherein the subject is a pediatric subject or an infant subject.

131. The method of claim 112, wherein the anti-anaphylactic agent comprises epinephrine.
